SEQUENCE LISTING (14 sequences)

SEQ ID NO: 1 - RNA, 50 nt, Artificial Sequence
  Synthetic sequence
cacgccugugaaaccguacuaagucucccgugucuucuuaucaccaucag

SEQ ID NO: 2 - RNA, 50 nt, Artificial Sequence
  Synthetic sequence
gaguaggauuacagaguauaacauagaguauaauauagaguauacaauag

SEQ ID NO: 3 - RNA, 39 nt, Artificial Sequence
  Synthetic sequence
gugucaaggacggugacugcagaaagacccauggaaagg

SEQ ID NO: 4 - RNA, 42 nt, Artificial Sequence
  Synthetic sequence
ccuaaacacaugugaaauagugucauaagcacaugggucaca

SEQ ID NO: 5 - RNA, 39 nt, Artificial Sequence
  Synthetic sequence
guagaggaggauaacaacacauauaaguauccguccacc

SEQ ID NO: 6 - RNA, 48 nt, Artificial Sequence
  Synthetic sequence
ugauggugauaagaagacacgggagacuuaguacgguuucacaggcgu

SEQ ID NO: 7 - DNA, 15 bp, Artificial Sequence
  Synthetic sequence
atgtcctggcagaac

SEQ ID NO: 8 - DNA, 18 bp, Artificial Sequence
  Synthetic sequence
catcctcccatcatatta

SEQ ID NO: 9 - DNA, 18 bp, Artificial Sequence
  Synthetic sequence
ggatgccccggggagagg

SEQ ID NO: 10 - RNA, 81 nt, Human Cytomegalovirus
accaguaaacuauuguauauauauaucaguuacuguuauggaucccacgucacuauuguauacucuauauuauacucuaug

SEQ ID NO: 11 - RNA, 89 nt, Human Cytomegalovirus
uuauacucuguaauccuacucaauaaacgugucacgccugugaaaccguacuaagucucccgugucuucuuaucaccaucaggugacau

SEQ ID NO: 12 - RNA, 50 nt, Artificial Sequence
  Synthetic sequence
cugauggugauaagaagacacgggagacuuaguacgguuucacaggcgug

SEQ ID NO: 13 - RNA, 18 nt, Artificial Sequence
  Synthetic sequence
ucucccgugucuucuuau

SEQ ID NO: 14 - RNA, 18 nt, Artificial Sequence
  Synthetic sequence
auaagaagacacgggaga

(19) United States
(12) Patent Application Publication
(10) Pub. No.: US 2024/0263180 A1
(43) Pub. Date: Aug. 8, 2024

(54) SMALL COMPLEMENTARY NUCLEIC ACIDS, COMPOSITIONS CONTAINING THE SAME, AND METHODS FOR USE AS ANTIVIRALS

(71) Applicant: HOWARD UNIVERSITY, Washington, DC (US)

(72) Inventors: Qiyi TANG, Washington, DC (US); Hua ZHU, Newark, NJ (US)

(73) Assignee: HOWARD UNIVERSITY, Washington, DC (US)

(21) Appl. No.: 18/564,938

(22) PCT Filed: May 27, 2022
(86) PCT No.: PCT/US2022/031327

§ 371 Date: Nov. 28, 2023

Related U.S. Application Data
(60) Provisional application No. 63/194,326, filed on May 28, 2021.

(51) Int. Cl. C12N 15/113; A61P 31/22

(52) U.S. Cl. CPC C12N 15/1131; A61P 31/22; C12N 2310/11; C12N 2320/33

(57) ABSTRACT

The present disclosure provides small complementary RNAs (scRNAs), compositions containing the same, and methods for using such small complementary RNAs as antivirals. The present disclosure provides, in more specific embodiments, scRNAs that are single-stranded and which are about 20-30 nucleotides (nt) long, and which are complementary to the intron of an essential viral gene, such as the major immediate early (MIE) gene of human cytomegalovirus (HCMV). Also provided herein are pharmaceutical compositions additionally containing a pharmaceutically acceptable carrier system, wherein the carrier system includes a cationic polymer which releases the scRNA in response to endosomal pH.

### STATEMENT REGARDING FEDERALLY SPONSORED RESEARCH OR DEVELOPMENT

This invention was partially supported by National Institutes of Health Grant number SC1112785. The U.S. government has certain rights to the invention.

### SEQUENCE LISTING

The present application contains a Sequence Listing that is hereby incorporated by reference in its entirety.

### TECHNICAL FIELD

The present disclosure relates generally to small complementary nucleic acids, such as, but not limited to, RNAs (scRNAs); compositions containing the same; and methods for using such small complementary nucleic acids, including scRNAs, as antivirals.

The present disclosure also provides, in more specific embodiments, small complementary nucleic acids, including scRNAs, that are single-stranded, that are about 20-30 nucleotides (nt) long, and that are complementary to the intron of an essential viral gene (such as, for example, the major immediate early (MIE) gene of human cytomegalovirus (HCMV)).

Also provided herein are pharmaceutical compositions containing such small complementary nucleic acids, including scRNAs, which additionally contain a pharmaceutically acceptable carrier system. In more specific embodiments, the carrier system includes a cationic polymer which releases the scRNA in response to endosomal pH.

### BACKGROUND

Human cytomegalovirus (HCMV) infection is the leading infectious cause of congenital disorders in newborns. Congenital HCMV infection causes permanent neurological and neurocognitive disabilities, and results in significant health problems worldwide. See Kirby et al. (“Congenital cytomegalovirus-a neglected health problem,” Lancet Infect. Dis., 2016, 16(8): 900-1); Boeckh et al. (“Cytomegalovirus: pathogen, paradigm, and puzzle,” J. Clin. Invest., 2011, 121(5): 1673-80); Cannon et al. (“Congenital cytomegalovirus (CMV) epidemiology and awareness,” J. Clin. Virol., 2009, 46 Suppl 4: S6-10); Bale et al. (“Fetal infections and brain development,” Clin. Perinatol., 2009, 36(3): 639-53); and Britt et al. (“Controversies in the natural history of congenital human cytomegalovirus infection: the paradox of infection and disease in offspring of women with immunity prior to pregnancy,” Med. Microbiol. Immunol., 2015, 204(3): 263-71). HCMV infects a large proportion of the population in general, and is responsible for significant morbidity and mortality in immunocompromised individuals. See Sweet et al. (“The pathogenicity of cytomegalovirus,” FEMS Microbiol. Rev., 1999, 23(4): 457-82); Landolfo et al. (“The human cytomegalovirus,” Pharmacol. Ther., 2003, 98(3): 269-97); and Mocarski et al. (“Cytomegaloviruses,” 2006, 5th Edition, D. M. Knipe and P. M. Howley (ed.), Philadelphia: Lippincott Williams & Wilkins). Currently, no vaccine is available.

Commonly-used treatments for HCMV infection include the synthetic acyclic analogue of 2′-deoxy-guanosine (and its derivatives) which targets viral DNA replication; and low molecular weight compounds that target the CMV terminase complex. See Bowman et al. (“Letermovir for the management of cytomegalovirus infection,” Expert Opin. Investig. Drugs., 2017, 26(2): 235-41); Chemaly et al. (“Letermovir for cytomegalovirus prophylaxis in hematopoietic-cell transplantation,” N. Engl. J. Med., 2014, 370(19): 1781-9); and Piret et al. (“Clinical development of letermovir and maribavir: Overview of human cytomegalovirus drug resistance,” Antiviral Res., 2019, 163: 91-105). However, drug resistance frequently occurs with these treatments, and additionally, none of them are suitable for foetal HCMV exposure. See Chou et al. (“Cytomegalovirus UL97 mutations in the era of ganciclovir and maribavir,” Rev. Med. Virol., 2008, 18(4): 233-46); Pass et al. (“Congenital cytomegalovirus infection: impairment and immunization,” J. Infect. Dis., 2007, 195(6): 767-9); and Cherrier et al. (“Emergence of letermovir resistance in a lung transplant recipient with ganciclovir-resistant cytomegalovirus infection,” Am. J. Transplant., 2018, 18(12): 3060-4). Accordingly, there remains an urgent need for new and specific inhibitors of HCMV replication, and especially for use in congenital CMV infection.

CMV replication in permissive host cells is a well-defined sequential process, including the following events (in chronological order): entry into cells; immediate-early (IE) gene expression; early (E) gene expression; DNA replication; late (L) gene expression; and viral production. See Mocarski et al. (“Cytomegaloviruses,” 2006, 5th Edition, D. M. Knipe and P. M. Howley (ed.), Philadelphia: Lippincott Williams & Wilkins). MIE genes are the most abundantly expressed viral genes at the IE stage of infection, and give rise to several nuclear phosphoproteins, the most abundant of which are the immediate-early proteins 1 (IE1, also known as IE72) and 2 (IE2, also known as IE86). See Tang et al. (“Mouse cytomegalovirus early M112/113 proteins control the repressive effect of IE3 on the major immediate-early promoter,” J. Virol., 2005, 79(1): 257-63); Tang et al. (“Immediate Early Interactions and Epigenetic Defense Mechanisms,” in “Cytomegaloviruses: Molecular Biology and Immunology,” Hethersett, Norwich, U. K.: Horizon Scientific Press, 2005); Hagemeier et al. (“The 72K IE1 and 80K IE2 proteins of human cytomegalovirus independently trans-activate the c-fos, c-myc and hsp70 promoters via basal promoter elements,” J. Gen. Virol., 1992, 73: 2385-93); Liu et al. (“A cis-acting element in the major immediate-early (IE) promoter of human cytomegalovirus is required for negative regulation by IE2,” J. Virol., 1991, 65(2): 897-903); Scully et al. (“The human cytomegalovirus IE2 86-kilodalton protein interacts with an early gene promoter via site-specific DNA binding and protein-protein associations,” J. Virol., 1995, 69(10): 6533-40); Awasthi et al. (“Analysis of splice variants of the immediate-early 1 region of human cytomegalovirus,” J. Virol., 2004, 78(15): 8191-200); Sadanari et al. (“The major immediate-early genes of human cytomegalovirus induce two novel proteins with molecular weights of 91 and 102 kilodaltons,” Arch. Virol., 2000, 145(6): 1257-66); Ahn et al. (“The major immediate-early proteins IE1 and IE2 of human cytomegalovirus colocalize with and disrupt PML-associated nuclear bodies at very early times in infected permissive cells,” J. Virol., 1997, 71(6): 4599-613); Meier et al. (“Effect of a modulator deletion on transcription of the human cytomegalovirus major immediate-early genes in infected undifferentiated and differentiated cells,” J. Virol., 1997, 71(2): 1246-55); Stenberg et al. (“The human cytomegalovirus major immediate-early gene,”Intervirology, 1996, 39(5-6): 343-9); and Stenberg et al. (“Structural analysis of the major immediate early gene of human cytomegalovirus,” J. Virol., 1984, 49(1): 190-9).

The expression of both IE1 and IE2 is under the control of the same promoter (MIEP). The MIE gene consists of 5 exons and 4 introns; IE1 and IE2 share the first 3 exons, but differ with respect to the last one (which is exon 4 for IE1, and exon 5 for IE2). The first exon does not encode any amino acids, but is related to initiation sequences. See Stenberg et al. (“The human cytomegalovirus major immediate-early gene,”Intervirology, 1996, 39(5-6): 343-9); and Stenberg et al. (“Structural analysis of the major immediate early gene of human cytomegalovirus,” J. Virol., 1984, 49(1): 190-9). The first intron (intron A) is believed to participate in MIE gene regulation via interaction with NF1 and CTCF. See Hennighausen et al. (“Nuclear factor 1 interacts with five DNA elements in the promoter region of the human cytomegalovirus major immediate early gene,” EMBO J., 1986, 5(6): 1367-71); and Martinez et al. (“CTCF Binding to the First Intron of the Major Immediate-Early (MIE) Gene of Human Cytomegalovirus (HCMV) Negatively Regulates MIE Gene Expression and HCMV Replication,” J. Virol., 2014, doi: JVI.00845-14).

HCMV is a member of the Herpesviridae, a family of large DNA viruses. Herpesviruses may encode as many as 200 different genes. The expression of some essential genes depends on RNA splicing after transcription; for example, the MIE gene of human cytomegalovirus (HCMV) gene has 5 exons, and 4 introns that need to be spliced to produce IE and IE2 (IE and IE2 are essential for viral infection and replication).

RNA splicing requires interaction of the intron RNA with cellular factors. The regulation of expression of the MIE gene at the transcriptional level has been studied extensively. See Meier et al. (“Effect of a modulator deletion on transcription of the human cytomegalovirus major immediate-early genes in infected undifferentiated and differentiated cells,” J. Virol., 1997, 71(2): 1246-55); Adair et al. (“Alteration of cellular RNA splicing and polyadenylation machineries during productive human cytomegalovirus infection,” J. Gen. Virol., 2004, 85: 3541-53); Akter et al. (“Two novel spliced genes in human cytomegalovirus,” J. Gen. Virol., 2003, 84: 1117-22); Sourvinos et al. (“Recruitment of human cytomegalovirus immediate-early 2 protein onto parental viral genomes in association with ND10 in live-infected cells,” J. Virol., 2007, 81(18): 10123-36); and Sinclair et al. (“Chromatin structure regulates human cytomegalovirus gene expression during latency, reactivation and lytic infection,” Biochim. Biophys. Acta., 2010, 1799(3-4): 286-95). However, the details of HCMV gene-splicing regulation still remain unclear.

Since polypyrimidine tract (Py)-binding protein (PTB) interferes with MIE gene splicing, see Cosme et al. (“Roles of polypyrimidine tract binding proteins in major immediate-early gene expression and viral replication of human cytomegalovirus,” J. Virol., 2009, 83(7): 2839-50); and since HCMV infection causes a temporal change of PTB, see Gaddy et al. (“Regulation of the subcellular distribution of key cellular RNA-processing factors during permissive human cytomegalovirus infection,” J. Gen. Virol., 2010, 91: 1547-59), the present inventors conceived of an antiviral mechanism in which small nucleic acids (such as RNA), complementary to an intron containing one or more Pys, could be used as an antiviral agent to inhibit virus replication (such as HCMV replication).

### SUMMARY OF THE INVENTION

The HCMV major immediate early (MIE) gene is essential for viral replication, and the most abundant products encoded by the MIE gene include IE1 and IE2. The IE1 and IE2 genes both share the MIE promoter (MIEP), as well as the first 3 exons and the first 2 introns. IE1 is expressed earlier than IE2 after CMV infection (or after MIE gene transfection). The present inventors identified two polypyrimidine (Py) tracts in intron 4 (between exons 4 and 5) responsible for transcriptional switching from IE1 to IE2. The present inventors further discovered that the first Py is important, and the second one is essential, for splicing and expression of IE2.

More specifically, the present inventors discovered that: (1) the second Py was essential for the IE2's fourth intron to bind to splicing factor (e.g., U2AF65); and (2) the first Py enhanced the binding of U2AF65 with the intron. The present inventors further discovered, using an HCMV BACmid with the second Py mutated, that the absence of the second Py completely abrogated virus production (while HCMV with the first Py mutated replicated with a defective phenotype).

In view of these discoveries, the present inventors conceived of small complementary nucleic acids (such as scRNAs), complementary to an intron RNA containing one or more Pys, as an antiviral. Such molecules are advantageous as antivirals because their inhibitory effect is specific to the virus (since it is complementary to the intron sequence, and not complementary to host cell sequences).

The present disclosure further provides small complementary nucleic acids (such as scRNAs) in combination with short interfering RNAs (siRNAs), to provide synergistic antiviral compositions and methods.

The present disclosure further provides delivery systems for efficient delivery of the small complementary nucleic acids (such as scRNAs) and compositions of the present disclosure.

Non-limiting embodiments of the disclosure include as follows.
[1] A single-stranded nucleic acid molecule comprising a sequence complementary to all or a part of an intron sequence of a viral mRNA containing one or more polypyrimidine (Py) tracts, wherein said nucleic acid molecule is capable of hybridizing to a region of said intron sequence containing one or more of said polypyrimidine (Py) tracts.
[2] The nucleic acid molecule of [1], wherein said viral mRNA is from the HCMV MIE gene.
[3] The nucleic acid molecule of [2], wherein the intron sequence is the fourth intron of the HCMV MIE gene.
[4] The nucleic acid molecule of [3], wherein said nucleic acid molecule, when introduced into HCMV-infected cells, is capable of inhibiting splicing and expression of HCMV IE2.
[5] The nucleic acid molecule of [1], wherein said sequence complementary to all or a part of the intron sequence is between 10-100 nucleotides in length.
[6] The nucleic acid molecule of [5], wherein said sequence complementary to all or a part of the intron sequence is between 10-50 nucleotides in length.
[7] The nucleic acid molecule of [1], wherein said sequence complementary to all or a part of the intron sequence comprises the sequence 5′-cacgccugugaaaccguacuaagucucccgugucuucuuaucaccaucag-3′.
[8] The nucleic acid molecule of [1], wherein said sequence complementary to all or a part of the intron sequence comprises a sequence having 90% or more sequence identity to the sequence 5′-cacgccugugaaaccguacuaagucucccgugucuucuuaucaccaucag-3′.
[9] A composition comprising the nucleic acid molecule of any one of [1]-[8], wherein said composition further comprises a delivery vehicle.
[10] The composition of [9], wherein the delivery vehicle comprises a polymer.
[11] The composition of [10], wherein the polymer comprises a cationic polymer.
[12] The composition of [10] or [11], wherein the delivery vehicle is a delivery particle.
[13] The composition of [12], wherein said delivery particle comprises a core structure comprising said nucleic acid molecule and at least one polymer, and wherein said delivery particle further comprises at least one wrapping layer that envelops said core structure.
[14] The composition of [13], wherein said core structure comprises said nucleic acid molecule, polyethylene imine (PEI), and polyspermine-imidazole-4,5-imine (PSI).
[15] The composition of [13] or [14], wherein said at least one wrapping layer comprises a hydrophilic polymer.
[16] The composition of [15], wherein said hydrophilic polymer is polyethylene glycol.
[17] The composition of [12], wherein said delivery particle comprises a targeting moiety.
[18] The composition of [17], wherein said targeting moiety targets said delivery particle to HCMV-infected cells.
[19] The composition of [18], wherein said targeting moiety is CX3CL1, or a mutant or derivative thereof.
[20] The nucleic acid of any of [1]-[8], wherein the nucleic acid molecule contains at least one of a modified base, a base analog, and an abasic site.
[21] The nucleic acid of any of [1]-[8], wherein the nucleic acid molecule contains DNA, RNA, or DNA and RNA.
[22] The nucleic acid of [21], wherein the nucleic acid molecule is a short complementary RNA (scRNA).
[23] The nucleic acid molecule of any of one [1]-[8] and [20]-[22], wherein the nucleic acid molecule is conjugated to a heterologous molecule.
[24] A method for treating a herpesvirus infection, preferably HCMV infection, comprising administering to a subject in need thereof the nucleic acid molecule of any one of [1]-[8] and [20]-[22].
[25] A method for treating a herpesvirus infection, preferably HCMV infection, comprising administering to a subject in need thereof the composition of any one of [9]-[19].
[26] A method of inhibiting transcriptional switching, comprising contacting the nucleic acid of any of [1]-[8] and [20]-[22] with a viral mRNA.

### INCORPORATION BY REFERENCE

All patents, publications, and patent applications cited in the present specification are herein incorporated by reference as if each individual patent, publication, or patent application was specifically and individually indicated to be incorporated by reference in its entirety for all purposes.

### BRIEF DESCRIPTION OF THE FIGURES

The features of the invention are set forth with particularity in the appended claims. A better understanding of the features and advantages of the present invention will be obtained by reference to the following detailed description that sets forth illustrative embodiments, in which the principles of the invention are utilized, and the accompanying figures. The figures are not proportionally rendered, nor are they to scale. The locations of indicators are approximate.

FIGS. 1A-1D show the results of experiments confirming the identity of the polypyrimidine tract (Py) for splicing of exon 5. FIG. 1A depicts the MIE RNA sequence between exons 4 and 5; the highlighted aauaaa sequence is the polyA signal of IE1, and the two predicted Py sequences are underlined (A-ucuccc and B-ucuucuu). FIG. 1B depicts the results of a Western blot assay to determine IE1/IE2 production. HEK 293T cells were transfected with pSVH, pSVHdPyA, pSVHdPyB, or pSVHdPyAB for 24 hours, and the whole-cell lysates were collected and subjected to SDS-PAGE to examine IE1/IE2 production using anti-IE1/2 antibody (MAB810). The relevant bands show IE1, SUMO-IE1, IE2, or SUMO-IE2, as indicated. FIG. 1C depicts the results of Real-time RT-PCR assay to determine IE1/IE2 expression. HEK 293T cells were transfected with pSVH, pSVHdPyA, pSVHdPyB, or pSVHdPyAB for 20 hours, and total RNA was isolated. One μg total RNA was used for real-time PCR to examine IE1/IE2 expression. The data in the graph represents the mean±standard deviation from 3 independent experiments. FIG. 1D depicts the results of an immunofluorescent assay (IFA) to determine IE2 and IE2 expression and localization. pSVH, pSVHdPyA, pSVHdPyB, or pSVHdPyAB, was transfected into MRC-5 cells for 20 hours, and the cells were then fixed and permeabilized to stain for IE1 (shown in green), IE2 (shown in red) and DAPI (shown in blue).

FIGS. 2A-2B show the results of experiments confirming interaction of the polypyrimidine tract (Py) and splicing factors. FIG. 2A depicts the results of an Electrophoretic Mobility Shift Assay (EMSA) used to analyze U2AF65 binding to RNA oligonucleotide probes containing both wt PyA and PyB (wt), mutated PyA (dPyA), mutated PyB (dPyB), or both mutations together (dPyAB). The positions of the free probe and the bound probe are indicated on the left side of the image. FIG. 2B depicts the results of an RNA Chromatin Immunoprecipitation (RNA ChIP) assay with HEK 293T cells transfected with pSVH, pSVHdPyA, pSVHdPyB, or pSVHdPyAB, at 24 h post-transfection with antibodies specific to U2AF65, PTB, or control IgG. Quantitative reverse-transcription PCR (qRT-PCR) was used to quantify ChIP efficiency with specific primers in the regions indicated. The bar graph represents the mean percentage of the input for each ChIP from 3 independent PCRs±the standard deviation.

FIGS. 3A-3B show the results of IE1/IE2 splicing assay experiments. FIG. 3A is an illustration showing the predicted sizes of IE2 mRNA or pre-mRNA and the primers (pShort and pIE2) used for the RT-PCR. FIG. 3B depicts the results of an IE1/IE2 splicing assay, in which HEK 293T cells were transfected with pSVH, pSVHdPyA, pSVHdPyB, or pSVHdPyAB, for 20 hours and total RNA was isolated. One pg total RNA was used to perform an RT-PCR using the SuperScript® III One-Step RT-PCR system with Platinum® Taq DNA polymerase (Invitrogen cat #12574018) according to the manufacturer's protocol. Controls included: (1) total RNA was directly used as template for PCR to exclude the possibility of contamination of plasmid DNA; and (2) plasmid DNA was used as template for PCR to show the size of the DNA in the DNA vector.

FIGS. 4A-4D show the results of experiments confirming the importance of the Pys for viral replication. FIG. 4A depicts the results of immunofluorescence assay (IFA) to examine IE1 and IE2 production. MRC-5 cells were fixed at 20 hours post transfection with the BACmid as indicated, and IFA assays were performed to examine the IE1 (FITC) and IE2 (Texas red) production. FIG. 4B depicts a viral growth curve for wild-type, HCMVdPyA, and revertant, viruses. Viral growth was determined using a plaque assay on MRC-5 cells, with infection at a multiplicity of infection (MOI) of 0.1. Each experiment was performed in triplicate. Virus growth was calculated by analyzing plaque formation. The numbers of infectious virus particles presented in the growth curve represent the averages from 3 experiments. Data with error bars depict mean±standard deviation. FIG. 4C depicts the results of Western blot assay to examine viral protein production by HCMVdPyA or its revertant after infection in MRC-5 cells at an MOI of 0.5. FIG. 4D depicts the results of immunostaining to detect IE1 and IE2. Human fibroblast cells (MRC-5) were infected with HCMVdPyA or its revertant at an MOI of 0.5 for different time periods, and the cells were fixed at 12 hpi and 24 hpi for subsequent immunostaining using antibodies against IE1 (FITC) and IE2 (Texas red). IE1- and IE2-positive cells were counted by fluorescence microscopy. Data with error bars depict mean±standard deviation. The results confirmed that PyA is important for IE2 production in a viral infection system.

FIGS. 5A-5C show the results of experiments confirming the importance of the PyA for IE2 splicing, expression, and interaction of intron 4 with splicing factors. FIG. 5A depicts the results of an IE2 splicing assay, conducted using MRC-5 cells infected with HCMVdPyA or its revertant for 16 hours at an MOI of 0.1. FIG. 5B depicts the results of an RNA ChIP assay to determine the interaction of the intron 4 with gene splicing factors (PTB and U2AF65) in MRC-5 cells that were infected with HCMVdPyA or its revertant for 16 hours at an MOI of 1. FIG. 5C depicts the results of real-time RT-PCR assays to determine the IE1/IE2 mRNA levels at different times after infection of MRC-5 cells with HCMVdPyA or its revertant at an MOI of 0.5.

FIGS. 6A-6F show the results of experiments determining the effects of a small complementary RNA (scRNA) on IE2 gene expression. FIG. 6A depicts the sequences of the ssRNA probe and the complementary scRNAPy. FIG. 6B depicts the results of incubation of the ssRNA probe with scRNAPy, showing that they formed double-stranded RNA (as indicated by a larger size than ssRNA or scRNAPy). FIG. 6C depicts the results of an EMSA assay used to determine U2AF65 binding to RNA oligonucleotide probes in the absence or presence of scRNAPy. FIG. 6D illustrates a model of scRNAPy interference with IE2 gene splicing; scRNAPy forms dsRNA with the Py-containing intron to block U2AF65 interaction with Py. FIG. 6E depicts the results of Western blot assay used to determine IE2 production. An scRNA complementary to the Pys (scRNAPy, the RNA sequence is shown in FIG. 6A), or an scRNA complementary to the upstream of the Pys (scRNAupPy: gag uag gau uac aga gua uaa cau aga gua uaa uau aga gua uac aau ag), or a scrambled RNA (made from luciferase gene), and pSVH, were cotransfected into HEK 293T cells for 24 hours, and the whole cell lysates were used to examine IE2 production using anti-IE2 antibody. Tubulin was used as a sample-loading control. FIG. 6F depicts the results of real-time RT-PCR to examine IE2 mRNA level. scRNAPy, or scRNAupPy, or scrambled RNA, and pSVH were cotransfected into HEK 293T cells for 20 hours and total RNA was isolated. One pg total RNA was used for real-time RT-PCR to examine IE2 mRNA level. The bar graph represents the mean±standard deviation from 3 independent experiments.

FIGS. 7A-7D show the results of Western blot assays used to determine the silencing efficiency of siRNA and scRNA in plasmid derived IE1/2 proteins co-transfected with lipofectamine 2000 in 293T cells. FIG. 7A depicts the results of experiments in which siRNA IE2 and siRNA IE1/2 were transfected together with a PSVH plasmid. FIG. 7B depicts the results of experiments in which scRNA IE2 and plasmid were co-transfected into 293T cells. FIG. 7C depicts the results of experiments in which siRNA and scRNA were used in combination to silence IE1/2 protein. FIG. 7A depicts the results of experiments in which the silencing effect of siRNA and scRNA in combination was measured, using plasmid formulated in polyplex and polywraplex.

### DETAILED DESCRIPTION OF THE INVENTION

The HCMV major immediate early (MIE) gene, which is essential for viral replication, encodes the IE1 and IE2 gene products. The IE1 and IE2 genes share the MIE promoter (MIEP), the first 3 exons, and the first 2 introns. As discussed above, the present inventors identified two polypyrimidine (Py) tracts in intron 4 (between exons 4 and 5) responsible for transcriptional switching from IE1 to IE2. The present inventors discovered that the first Py is important, and that the second one is essential, for the splicing and expression of IE2.

In view of these discoveries, the present inventors conceived of small complementary nucleic acids (such as scRNAs), complementary to an intron RNA containing one or more Pys, that bind to the intron sequence and thereby interfere with gene splicing. The nucleic acids are preferably single-stranded, or contain at least a singles-stranded region to effect complementary binding. This mechanism is thus applicable to the inhibition of a variety of DNA viruses, such as herpesvirus and adenoviruses, for example.

The present disclosure provides, in certain embodiments, small complementary nucleic acids (such as scRNAs) that are complementary to all or a part of the intron RNA covering the two Pys in intron 4 of the HCMV MIE gene.

The present disclosure also provides compositions and methods in which small complementary nucleic acids (such as scRNAs) are combined with, or used in combination with, short interfering RNAs (siRNAs). This combination may thereby provide synergistic antiviral compositions and methods.

The present disclosure further provides delivery systems for efficient delivery of the small complementary nucleic acids (such as scRNAs) and compositions of the present disclosure.

It is to be understood that the terminology used herein is for the purpose of describing particular embodiments only and is not intended to be limiting. As used in the present specification and the appended claims, the singular forms “a,” “an,” and “the” include plural referents unless the context clearly dictates otherwise. Thus, for example, reference to “a polynucleotide” includes one or more polynucleotides, and reference to “a vector” includes one or more vectors.

Unless defined otherwise, all technical and scientific terms used herein have the same meaning as commonly understood by one of ordinary skill in the art to which the invention pertains. Although other methods and materials similar, or equivalent, to those described herein can be useful in the present invention, preferred materials and methods are described herein.

In view of the teachings of the present specification, one of ordinary skill in the art can apply conventional techniques of immunology, biochemistry, chemistry, molecular biology, microbiology, cell biology, genomics, and recombinant polynucleotides, as taught, for example, by the following standard texts: Abbas et al. (Cellular and Molecular Immunology, 2017, 9th Edition, Elsevier, ISBN 978-0323479783); Butterfield et al. (Cancer Immunotherapy Principles and Practice, 2017, 1st Edition, Demos Medical, ISBN 978-1620700976); Kenneth Murphy (Janeway's Immunobiology, 2016, 9th Edition, Garland Science, ISBN 978-0815345053); Stevens et al. (Clinical Immunology and Serology: A Laboratory Perspective, 2016, 4th Edition, Davis Company, ISBN 978-0803644663); E. A. Greenfield (Antibodies: A Laboratory Manual, 2014, Second edition, Cold Spring Harbor Laboratory Press, ISBN 978-1-936113-81-1); R. I. Freshney (Culture of Animal Cells: A Manual of Basic Technique and Specialized Applications, 2016, 7th Edition, Wiley-Blackwell, ISBN 978-1118873656); C. A. Pinkert (Transgenic Animal Technology, Third Edition: A Laboratory Handbook, 2014, Elsevier, ISBN 978-0124104907); H. Hedrich (The Laboratory Mouse, 2012, Second Edition, Academic Press, ISBN 978-0123820082); Behringer et al. (Manipulating the Mouse Embryo: A Laboratory Manual, 2013, Fourth Edition, Cold Spring Harbor Laboratory Press, ISBN 978-1936113019); McPherson et al. (PCR 2: A Practical Approach, 1995, IRL Press, ISBN 978-0199634248); J. M. Walker (Methods in Molecular Biology (Series), Humana Press, ISSN 1064-3745); Rio et al. (RNA: A Laboratory Manual, 2010, Cold Spring Harbor Laboratory Press, ISBN 978-0879698911); Methods in Enzymology (Series), Academic Press; Green et al. (Molecular Cloning: A Laboratory Manual, 2012, Fourth Edition, Cold Spring Harbor Laboratory Press, ISBN 978-1605500560); and G. T. Hermanson (Bioconjugate Techniques, 2013, Third Edition, Academic Press, ISBN 978-0123822390).

A “linker region sequence,” “linker sequence,” and “linker polynucleotide” are used interchangeably herein and refer to a sequence of one or more nucleotides covalently attached to a first nucleic acid sequence (e.g., 5′-linker nucleotide sequence-first nucleic acid sequence-3′). In some embodiments, a linker nucleotide sequence connects two separate nucleic acid sequences to form a single polynucleotide (e.g., 5′-first nucleic acid sequence-linker nucleotide sequence-second nucleic acid sequence-3′). Other examples of linker sequences include, but are not limited to, 5′-first nucleic acid sequence-linker nucleotide sequence-3′, and 5′-linker nucleotide sequence-first first nucleic acid sequence-linker nucleotide sequence-3′. In some embodiments, the linker nucleotide sequence can be a single-stranded nucleotide sequence of unpaired nucleic acid bases that do not interact with each other through hydrogen bond formation to create a secondary structure. In some embodiments, a linker element nucleotide sequence can be about 100 or less, about 90 or less, about 80 or less, about 70 or less, about 60 or less, about 50 or less, about 40 or less, about 30 or less, about 20 or less, about 15 or less, about 14 or less, about 13 or less, about 12 or less, about 11 or less, about 10 or less, about 9 or less, about 8 or less, about 7 or less, about 6 or less, about 5 or less, about 4 or less, about 3 or less, or about 2 or less, bases in length.

The terms “wild-type,” “naturally occurring,” and “unmodified” are used herein to mean the typical (or most common) form, appearance, phenotype, or strain existing in nature; for example, the typical form of cells, organisms, polynucleotides, proteins, macromolecular complexes, genes, RNAs, DNAs, or genomes as they occur in, and can be isolated from, a source in nature. The wild-type form, appearance, phenotype, or strain serve as the original parent before an intentional modification. Thus, mutant, variant, engineered, recombinant, and modified forms are not wild-type forms.

By “isolated” is meant, when referring to a polypeptide, that the indicated molecule is separate and discrete from the whole organism with which the molecule is found in nature or is present in the substantial absence of other biological macromolecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The term “purified” as used herein preferably means at least 75% by weight, more preferably at least 85% by weight, more preferably still at least 95% by weight, and most preferably at least 98% by weight, of the same molecule is present.

The terms “engineered,” “genetically engineered,” “genetically modified,” “recombinant,” “modified,” “non-naturally occurring,” and “non-native” indicate intentional human manipulation of the genome of an organism or cell. The terms encompass methods of genomic modification that include genomic editing, as defined herein, as well as techniques that alter gene expression or inactivation, enzyme engineering, directed evolution, knowledge-based design, random mutagenesis methods, gene shuffling, codon optimization, and the like. Methods for genetic engineering are known in the art.

“Covalent bond,” “covalently attached,” “covalently bound,” “covalently linked,” “covalently connected,” and “molecular bond” are used interchangeably herein and refer to a chemical bond that involves the sharing of electron pairs between atoms. Examples of covalent bonds include, but are not limited to, phosphodiester bonds and phosphorothioate bonds.

“Non-covalent bond,” “non-covalently attached,” “non-covalently bound,” “non-covalently linked,” “non-covalent interaction,” and “non-covalently connected” are used interchangeably herein, and refer to any relatively weak chemical bond that does not involve sharing of a pair of electrons. Multiple non-covalent bonds often stabilize the conformation of macromolecules and mediate specific interactions between molecules. Examples of non-covalent bonds include, but are not limited to hydrogen bonding, ionic interactions (e.g., Na⁺Cl⁻), van der Waals interactions, and hydrophobic bonds.

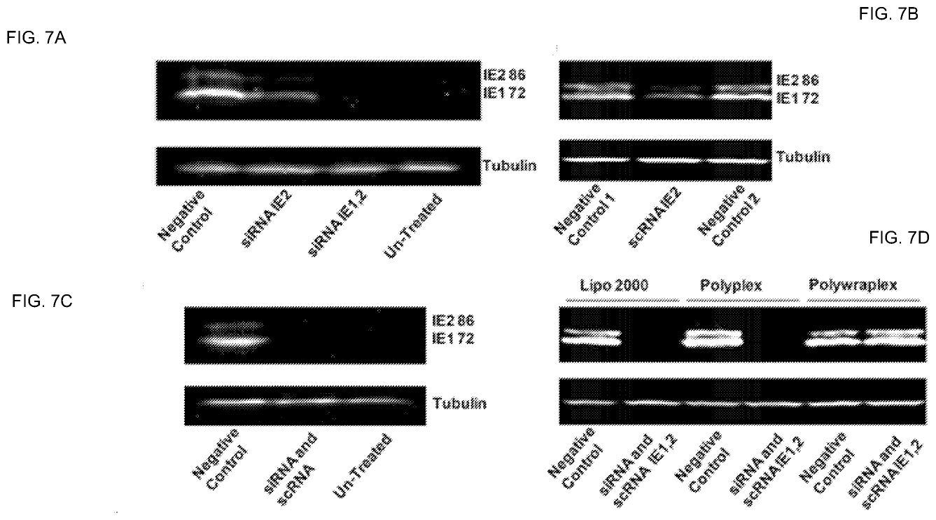

As used herein, “hydrogen bonding,” “hydrogen-base pairing,” and “hydrogen bonded” are used interchangeably and refer to canonical hydrogen bonding and non-canonical hydrogen bonding including, but not limited to, “Watson-Crick-hydrogen-bonded base pairs” (W—C-hydrogen-bonded base pairs or W—C hydrogen bonding); “Hoogsteen-hydrogen-bonded base pairs” (Hoogsteen hydrogen bonding); and “wobble-hydrogen-bonded base pairs” (wobble hydrogen bonding). W—C hydrogen bonding, including reverse W—C hydrogen bonding, refers to purine-pyrimidine base pairing, that is, adenine:thymine, guanine:cytosine, and uracil:adenine. Hoogsteen hydrogen bonding, including reverse Hoogsteen hydrogen bonding, refers to a variation of base pairing in nucleic acids wherein two nucleobases, one on each strand, are held together by hydrogen bonds in the major groove. This non-W—C hydrogen bonding can allow a third strand to wind around a duplex and form triple-stranded helices. Wobble hydrogen bonding, including reverse wobble hydrogen bonding, refers to a pairing between two nucleotides in RNA molecules that does not follow Watson-Crick base pair rules. There are four major wobble base pairs: guanine:uracil, inosine (hypoxanthine):uracil, inosine:adenine, and inosine:cytosine. Wobble base interaction are also known to occur between inosine:thymine and inosine:guanine. Inosine bases and deoxy inosine bases can be referred to as “universal pairing bases” as they are capable of hydrogen bonding with the canonical DNA and RNA bases (see, e.g., FIG. 7). See also Watkins et al. (Nucleic Acid Research, 2005, 33(19):6258-67). Rules for canonical hydrogen bonding and non-canonical hydrogen bonding are known to those of ordinary skill in the art. See, e.g., R. F. Gesteland (The RNA World, Third Edition (Cold Spring Harbor Monograph Series), 2005, Cold Spring Harbor Laboratory Press, ISBN 978-0879697396); R. F. Gesteland (The RNA World, Second Edition (Cold Spring Harbor Monograph Series), 1999, Cold Spring Harbor Laboratory Press, ISBN 978-0879695613); R. F. Gesteland (The RNA World, First Edition (Cold Spring Harbor Monograph Series), 1993, Cold Spring Harbor Laboratory Press, 978-0879694562) (see, e.g., Appendix 1: Structures of Base Pairs Involving at Least Two Hydrogen Bonds, I. Tinoco); W. Saenger (Principles of Nucleic Acid Structure, 1988, Springer International Publishing AG, ISBN 978-0-387-90761-1); S. Neidle (Principles of Nucleic Acid Structure, 2007, First Edition, Academic Press, ISBN 978-01236950791).

“Connect,” “connected,” and “connecting” are used interchangeably herein, and refer to a covalent bond or a non-covalent bond between two macromolecules (e.g., polynucleotides, proteins, and the like).

As used herein, the terms “nucleic acid sequence,” “nucleotide sequence,” and “oligonucleotide” are interchangeable and refer to a polymeric form of nucleotides. As used herein, the term “polynucleotide” refers to a polymeric form of nucleotides that has one 5′ end and one 3′ end and can comprise one or more nucleic acid sequences. The nucleotides may be deoxyribonucleotides (DNA), ribonucleotides (RNA), analogs thereof, or combinations thereof, and may be of any length. Polynucleotides may perform any function and may have various secondary and tertiary structures. The terms encompass known analogs of natural nucleotides and nucleotides that are modified in the base, sugar, and/or phosphate moieties. Analogs of a particular nucleotide have the same base-pairing specificity (e.g., an analog of A base pairs with T). A polynucleotide may comprise one modified nucleotide or multiple modified nucleotides. Examples of modified nucleotides include fluorinated nucleotides, methylated nucleotides, and nucleotide analogs. Nucleotide structure may be modified before or after a polymer is assembled. Following polymerization, polynucleotides may be additionally modified via, for example, conjugation with a labeling component or target binding component. A nucleotide sequence may incorporate non-nucleotide components. The terms also encompass nucleic acids comprising modified backbone residues or linkages, that are synthetic, naturally occurring, and/or non-naturally occurring, and have similar binding properties as a reference polynucleotide (e.g., DNA or RNA). Examples of such analogs include, but are not limited to, phosphorothioates, phosphoramidates, methyl phosphonates, chiral-methyl phosphonates, 2-O-methyl ribonucleotides, peptide-nucleic acids (PNAs), Locked Nucleic Acid (LNA™) (Exiqon, Woburn, MA) nucleosides, glycol nucleic acid, bridged nucleic acids, and morpholino structures.

As used herein, the terms “abasic,” “abasic site,” “abasic nucleotide,” and “apurinic/apyrimidinic site,” are used interchangeably and refer to a site in a nucleotide sequence that lacks the purine or a pyrimidine base. In certain embodiments, abasic sites comprise a deoxyribose site. In other embodiments, abasic sites comprise a ribose site. In yet further embodiments, abasic sites comprise a modified backbone, such as a pentose ring with a 1′ hydroxyl group. An abasic site cannot form hydrogen base pair bonding with a complementary nitrogen base of a DNA or RNA nucleotide, because it does not contain a nitrogen base.

As used herein, the term “base analog” refers to a compound having structural similarity to a canonical purine or pyrimidine base occurring in DNA or RNA. The base analog may contain a modified sugar and/or a modified nucleobase, as compared to a purine or pyrimidine base occurring naturally in DNA or RNA. In some embodiments, the base analog is inosine or deoxyinosine, such as 2′-deoxyinosine. In other embodiments, the base analog is a 2′-deoxyribonucleoside, 2′-ribonucleoside, 2′-deoxyribonucleotide or a 2′-ribonucleotide, wherein the nucleobase includes a modified base (such as, for example, xanthine, uridine, oxanine (oxanosine), 7-methlguanosine, dihydrouridine, 5-methylcytidine, C3 spacer, 5-methyl dC, 5-hydroxybutynl-2′-deoxyuridine, 5-nitroindole, 5-methyl iso-deoxycytosine, iso deoxyguanosine, deoxyuradine, iso deoxycytidine, other 0-1 purine analogs, N-6-hydroxylaminopurine, nebularine, 7-deaza hypoxanthine, other 7-deazapurines, and 2-methyl purines). In some embodiments, the base analog may be selected from the group consisting of 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine. The term “base analog” also includes, for example, 2′-deoxyribonucleosides, 2′-ribonucleosides, 2′-deoxyribonucleotides or 2′-ribonucleotides, wherein the nucleobase is a substituted hypoxanthine. For instance, the substituted hypoxanthine may be substituted with a halogen, such as fluorine or chlorine. In some embodiments, the base analog may be a fluoroinosine or a chloroinosine, such as 2-chloroinosine, 6-chloroinosine, 8-chloroinosine, 2-fluoroinosine, 6-fluoroinosine, or 8-fluoroinosine. In other embodiments, the base analog is deoxyuradine. In other embodiments the base analog is a nucleic acid mimic (such as, for example, artificial nucleic acids and xeno nucleic acids (XNA).

Peptide-nucleic acids (PNAs) are synthetic homologs of nucleic acids wherein the polynucleotide phosphate-sugar backbone is replaced by a flexible pseudo-peptide polymer. Nucleobases are linked to the polymer. PNAs have the capacity to hybridize with high affinity and specificity to complementary sequences of RNA and DNA.

In phosphorothioate nucleic acids, the phosphorothioate (PS) bond substitutes a sulfur atom for a non-bridging oxygen in the polynucleotide phosphate backbone. This modification makes the internucleotide linkage resistant to nuclease degradation. In some embodiments, phosphorothioate bonds are introduced between the last 3 to 5 nucleotides at the 5′-end or 3′-end sequences of a polynucleotide sequence to inhibit exonuclease degradation. Placement of phosphorothioate bonds throughout an entire oligonucleotide helps reduce degradation by endonucleases as well.

Threose nucleic acid (TNA) is an artificial genetic polymer. The backbone structure of TNA comprises repeating threose sugars linked by phosphodiester bonds. TNA polymers are resistant to nuclease degradation. TNA can self-assemble by base-pair hydrogen bonding into duplex structures.

Linkage inversions can be introduced into polynucleotides through use of “reversed phosphoramidites” (see, e.g., www.ucalgary.ca/dnalab/synthesis/-modifications/linkages). A 3′-3′ linkage at a terminus of a polynucleotide stabilizes the polynucleotide to exonuclease degradation by creating an oligonucleotide having two 5′-OH termini but lacking a 3′-OH terminus. Typically, such polynucleotides have phosphoramidite groups on the 5′-OH position and a dimethoxytrityl (DMT) protecting group on the 3′-OH position. Normally, the DMT protecting group is on the 5′-OH and the phosphoramidite is on the 3′-OH.

Polynucleotide sequences are displayed herein in the conventional 5′ to 3′ orientation unless otherwise indicated.

As used herein, “sequence identity” generally refers to the percent identity of nucleotide bases or amino acids comparing a first polynucleotide or polypeptide to a second polynucleotide or polypeptide using algorithms having various weighting parameters. Sequence identity between two polynucleotides or two polypeptides can be determined using sequence alignment by various methods and computer programs (e.g., BLAST, CS-BLAST, FASTA, HMMER, L-ALIGN, and the like) available through the worldwide web at sites including, but not limited to, GENBANK (www.ncbi.nlm.nih.gov/genbank/) and EMBL-EBI (www.ebi.ac.uk.). Sequence identity between two polynucleotides or two polypeptide sequences is generally calculated using the standard default parameters of the various methods or computer programs. A high degree of sequence identity between two polynucleotides or two polypeptides is typically between about 90% identity and 100% identity over the length of the reference polypeptide, for example, about 90% identity or higher, preferably about 95% identity or higher, more preferably about 98% identity or higher. A moderate degree of sequence identity between two polynucleotides or two polypeptides is typically between about 80% identity to about 85% identity, for example, about 80% identity or higher, preferably about 85% identity over the length of the reference polypeptide. A low degree of sequence identity between two polynucleotides or two polypeptides is typically between about 50% identity and 75% identity, for example, about 50% identity, preferably about 60% identity, more preferably about 75% identity over the length of the reference polypeptide.

For instance, a nucleic acid sequence of the present disclosure may have a particular sequence identity to a reference sequence. This sequence identity may be, for example, 25% or more, 50% or more, 75% or more, 80% or more, 85% or more, 90% or more, 91% or more, 92% or more, 93% or more, 94% or more, 95% or more, 96% or more, 97% or more, 98% or more, or 99% or more.

As used herein, “hybridization,” “hybridize,” or “hybridizing” is the process of combining two complementary single-stranded DNA or RNA molecules so as to form a single double-stranded molecule (DNA/DNA, DNA/RNA, RNA/RNA) through hydrogen base pairing. Hybridization stringency is typically determined by the hybridization temperature and the salt concentration of the hybridization buffer; e.g., high temperature and low salt provide high stringency hybridization conditions. Examples of salt concentration ranges and temperature ranges for different hybridization conditions are as follows: high stringency, approximately 0.01M to approximately 0.05M salt, hybridization temperature 5° C. to 10° C. below Tm; moderate stringency, approximately 0.16M to approximately 0.33M salt, hybridization temperature 20° C. to 29° C. below Tm; and low stringency, approximately 0.33M to approximately 0.82M salt, hybridization temperature 40° C. to 48° C. below Tm. Tm of duplex nucleic acid sequences is calculated by standard methods well-known in the art. See, e.g., Maniatis et al. (Molecular Cloning: A Laboratory Manual, 1982, Cold Spring Harbor Laboratory Press: New York); Casey et al. (Nucleic Acids Research, 1977, 4:1539-1552); Bodkin et al. Journal of Virological Methods, 1985, 10(1):45-52); and Wallace et al. (Nucleic Acids Research, 1981, 9(4):879-894). Algorithm prediction tools to estimate Tm are also widely available. High stringency conditions for hybridization typically refer to conditions under which a polynucleotide complementary to a target sequence predominantly hybridizes with the target sequence, and substantially does not hybridize to non-target sequences. Typically, hybridization conditions are of moderate stringency, preferably high stringency.

As used herein, “complementarity” refers to the ability of a nucleic acid sequence to form hydrogen bonds with another nucleic acid sequence (e.g., through canonical Watson-Crick base pairing). A percent complementarity indicates the percentage of residues in a nucleic acid sequence that can form hydrogen bonds with a second nucleic acid sequence. If two nucleic acid sequences have 100% complementarity, the two sequences are perfectly complementary, i.e., all of the contiguous residues of a first polynucleotide hydrogen bond with the same number of contiguous residues in a second polynucleotide.

As used herein, “binding” refers to a non-covalent interaction between macromolecules (e.g., between a protein and a polynucleotide, between a polynucleotide and a polynucleotide, or between a protein and a protein, and the like). Such non-covalent interaction is also referred to as “associating” or “interacting” (e.g., if a first macromolecule interacts with a second macromolecule, the first macromolecule binds to second macromolecule in a non-covalent manner). Some portions of a binding interaction may be sequence-specific (the terms “sequence-specific binding,” “sequence-specifically bind,” “site-specific binding,” and “site specifically binds” are used interchangeably herein). Binding interactions can be characterized by a dissociation constant (Kd). “Binding affinity” refers to the strength of the binding interaction. An increased binding affinity is correlated with a lower Kd.

As used herein, the terms “regulatory sequences,” “regulatory elements,” and “control elements” are interchangeable and refer to polynucleotide sequences that are upstream (5′ non-coding sequences), within, or downstream (3′ non-translated sequences) of a polynucleotide target to be expressed. Regulatory sequences influence, for example, the timing of transcription, amount or level of transcription, RNA processing or stability, and/or translation of the related structural nucleotide sequence. Regulatory sequences may include activator binding sequences, enhancers, introns, polyadenylation recognition sequences, promoters, transcription start sites, repressor binding sequences, stem-loop structures, translational initiation sequences, internal ribosome entry sites (IRES), translation leader sequences, transcription termination sequences (e.g., polyadenylation signals and poly-U sequences), translation termination sequences, primer binding sites, and the like.

Regulatory elements include those that direct constitutive, inducible, and repressible expression of a nucleotide sequence in many types of host cells and those that direct expression of the nucleotide sequence only in certain host cells (e.g., tissue-specific regulatory sequences). In some embodiments, a vector comprises one or more pol III promoters, one or more pol II promoters, one or more pol I promoters, or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer; see, e.g., Boshart et al. (Cell, 1985, 41:521-530)), the SV40 promoter, the dihydrofolate reductase promoter, the β-actin promoter, the phosphoglycerol kinase (PGK) promoter, and the EF1α promoter. It will be appreciated by those skilled in the art that the design of an expression vector may depend on such factors as the choice of the host cell to be transformed, the level of expression desired, and the like. A vector can be introduced into host cells to thereby produce transcripts, proteins, or peptides, including fusion proteins or peptides, encoded by nucleic acid sequences as described herein.

“Gene” as used herein refers to a polynucleotide sequence comprising exons and related regulatory sequences. A gene may further comprise introns and/or untranslated regions (UTRs).

As used herein, the term “operably linked” refers to polynucleotide sequences or amino acid sequences placed into a functional relationship with one another. For example, regulatory sequences (e.g., a promoter or enhancer) are “operably linked” to a polynucleotide encoding a gene product if the regulatory sequences regulate or contribute to the modulation of the transcription of the polynucleotide. Operably linked regulatory elements are typically contiguous with the coding sequence. However, enhancers can function if separated from a promoter by up to several kilobases or more. Accordingly, some regulatory elements may be operably linked to a polynucleotide sequence but not contiguous with the polynucleotide sequence. Similarly, translational regulatory elements contribute to the modulation of protein expression from a polynucleotide.

As used herein, “expression” refers to transcription of a polynucleotide from a DNA template, resulting in, for example, a messenger RNA (mRNA) or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene products.” Expression may include splicing the mRNA in a eukaryotic cell, if the polynucleotide is derived from genomic DNA.

A “coding sequence” or a sequence that “encodes” a selected polypeptide, is a nucleic acid molecule that is transcribed (in the case of DNA) and translated (in the case of mRNA) into a polypeptide in vitro or in vivo when placed under the control of appropriate regulatory sequences. The boundaries of the coding sequence are determined by a start codon at the 5′ terminus and a translation stop codon at the 3′ terminus. A transcription termination sequence may be located 3′ to the coding sequence.

As used herein, the term “modulate” refers to a change in the quantity, degree or amount of a function. Thus, “modulation” of gene expression includes both gene activation and gene repression. Modulation can be assayed by determining any characteristic directly or indirectly affected by the expression of the target gene. Such characteristics include, for example, changes in RNA or protein levels, protein activity, product levels, expression of the gene, or activity level of reporter genes.

“Vector” and “plasmid” as used herein refer to a polynucleotide vehicle to introduce genetic material into a cell. Vectors can be linear or circular. Vectors can contain a replication sequence capable of effecting replication of the vector in a suitable host cell (e.g., an origin of replication). Upon transformation of a suitable host, the vector can replicate and function independently of the host genome or integrate into the host genome. Vector design depends, among other things, on the intended use and host cell for the vector, and the design of a vector of the invention for a particular use and host cell is within the level of skill in the art. The four major types of vectors are plasmids, viral vectors, cosmids, and artificial chromosomes. Typically, vectors comprise an origin of replication, a multicloning site, and/or a selectable marker. An expression vector typically comprises an expression cassette. By “recombinant virus” is meant a virus that has been genetically altered, e.g., by the addition or insertion of a heterologous nucleic acid construct into a viral genome or portion thereof.

As used herein, “expression cassette” refers to a polynucleotide construct generated using recombinant methods or by synthetic means and comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in a vector to form an expression vector.

As used herein, the term “between” is inclusive of end values in a given range (e.g., between about 1 and about 50 nucleotides in length includes 1 nucleotide and 50 nucleotides.

As used herein, the term “amino acid” refers to natural and synthetic (unnatural) amino acids, including amino acid analogs, modified amino acids, peptidomimetics, glycine, and D or L optical isomers.

As used herein, the terms “peptide,” “polypeptide,” and “protein” are interchangeable and refer to polymers of amino acids. A polypeptide may be of any length. It may be branched or linear, it may be interrupted by non-amino acids, and it may comprise modified amino acids. The terms also refer to an amino acid polymer that has been modified through, for example, acetylation, disulfide bond formation, glycosylation, lipidation, phosphorylation, pegylation, biotinylation, cross-linking, and/or conjugation (e.g., with a labeling component or ligand). Polypeptide sequences are displayed herein in the conventional N-terminal to C-terminal orientation, unless otherwise indicated. Polypeptides and polynucleotides can be made using routine techniques in the field of molecular biology. Furthermore, essentially any polypeptide or polynucleotide is available from commercial sources.

The terms “fusion protein” and “chimeric protein” as used herein refer to a single protein created by joining two or more proteins, protein domains, or protein fragments that do not naturally occur together in a single protein.

A “moiety” as used herein refers to a portion of a molecule. A moiety can be a functional group or describe a portion of a molecule with multiple functional groups (e.g., that share common structural aspects). The terms “moiety” and “functional group” are typically used interchangeably; however, a “functional group” can more specifically refer to a portion of a molecule that comprises some common chemical behavior. “Moiety” is often used as a structural description. In some embodiments, a 5′ terminus, a 3′ terminus, or a 5′ terminus and a 3′ terminus can comprise one or more moieties.

The terms “modified protein,” “mutated protein,” “protein variant,” and “engineering protein” as used herein typically refers to a protein that has been modified such that it comprises a non-native sequence (i.e., the modified protein has a unique sequence compared to an unmodified protein).

The terms “subject,” “individual,” or “patient” are used interchangeably herein and refer to any member of the phylum Chordata, including, without limitation, humans and other primates, including non-human primates, such as rhesus macaques, chimpanzees, and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys, and other gallinaceous birds, ducks, and geese; and the like. The term does not denote a particular age or gender. Thus, the term includes adult, young, and newborn individuals as well as males and females.

The terms “effective amount” or “therapeutically effective amount” of a composition or agent, refer to a sufficient amount of the composition or agent to provide the desired response. Preferably, the effective amount will prevent, avoid, or eliminate one or more harmful side-effects. The exact treatment amount required will vary from subject to subject, depending on the species, age, and general condition of the subject, the severity of the condition being treated, and the particular treatment used, mode of administration, and the like. An appropriate “effective” amount in any individual case may be determined by one of ordinary skill in the art using routine experimentation.

“Treatment” or “treating” a particular disease includes: (1) preventing the disease, for example, preventing the development of the disease or causing the disease to occur with less intensity in a subject that may be predisposed to the disease, but does not yet experience or display symptoms of the disease; (2) inhibiting the disease, for example, reducing the rate of development, arresting the development or reversing the disease state; and/or (3) relieving symptoms of the disease, for example, decreasing the number of symptoms experienced by the subject.

“Transformation” as used herein refers to the insertion of an exogenous polynucleotide into a host cell, irrespective of the method used for insertion. For example, transformation can be by direct uptake, transfection, infection, and the like. Nucleic acids and polynucleotides can be introduced into the cell using, for example, viral vectors, nucleofection, gene gun, sonoporation, cell squeezing, lipofection, or chemicals (e.g., cell penetrating peptides).

“Herpesvirus” as used herein refers to a member of the family Herpesviridae, and therefore includes, but is not limited to, viruses such as Herpes Simplex Virus-1 (HSV-1), Herpes Simplex Virus-2 (HSV-2), Varicella Zoster Virus (VZV), Epstein-Barr Virus (EBV), Human Cytomegalovirus (HCMV), Human Herpesviruses 6 and 7, and Kaposi's sarcoma-associated Herpesvirus (KSHV).

### Small Complementary Nucleic Acids

As described herein, small complementary nucleic acids (such as scRNAs) of the present disclosure can be designed and synthesized which inhibit DNA virus replication, such as herpesvirus and/or adenovirus replication. In some embodiments, the small complementary nucleic acids (such as scRNAs) may be complementary to an intron RNA in an immediate-early gene of a herpesvirus that contains one or more Pys. In certain embodiments, the small complementary nucleic acids (such as scRNAs) may be complementary to an intron RNA in an immediate-early gene of HCMV that contains one or more Pys.

In certain embodiments, the small complementary nucleic acids (such as scRNAs) may be complementary to all or a part of intron 4 (between exons 4 and 5) of the HCMV MIE gene, which includes two Py tracts responsible for transcriptional switching from IE1 to IE2.

In some embodiments, the scRNA complementary to the intron RNA may cover one or both of the Pys.

In embodiments, small complementary nucleic acids (such as scRNAs) of the present disclosure may interfere with interaction of a splicing factor with an intron, and preferably, repress expression of an essential gene thereby. In some embodiments thereof, the splicing factor is U2AF65 and/or U2AF35. In some embodiments thereof, the intron is intron 4 of the MIE gene of HCMV.

In some embodiments, the small complementary nucleic acid (such as scRNA) of the present disclosure comprises, consists of, is a fragment or portion thereof, or is a modified version of derivative thereof, of the sequence 5′-cacgccugugaaaccguacuaagucucccgugucuucuuaucaccaucag-3′.

In some embodiments, the small complementary nucleic acid is a DNA, an RNA, or a mixture of DNA and RNA, and may or may not include one or more abasic sites, base analogs, and/or modified bases, for example. In some embodiments, the small complementary nucleic acid sequence contains RNA. In some embodiments, the small complementary nucleic acid sequence contains only RNA. Additionally, siRNAs as contemplated herein may contain, in some embodiments, DNA bases.

In certain aspects and embodiments, nucleic acid molecules herein (including, but not limited to, small complementary nucleic acids and siRNAs) may include one or more modifications (or chemical modifications). Such modifications can be in the nucleotide sugar, nucleotide base, nucleotide phosphate group and/or the phosphate backbone of a polynucleotide.

In certain embodiments, modifications as disclosed herein may be used to increase the in vivo stability of nucleic acid molecules (such as small complementary nucleic acids, scRNAs and/or siRNAs), particularly the stability in serum, and/or to increase bioavailability of the molecules. Non-limiting examples of modifications include, without limitation, internucleotide or internucleoside linkages; deoxynucleotides or dideoxyribonucleotides at any position and strand of the nucleic acid molecule; nucleic acid (e.g., ribonucleic acid) with a modification at the 2′-position preferably selected from an amino, fluoro, methoxy, alkoxy and alkyl; 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, biotin group, and terminal glyceryl and/or inverted deoxy abasic residue incorporation, sterically hindered molecules, such as fluorescent molecules and the like. Other modified nucleotides may include, for example, 3′-deoxyadenosine, 3′-azido-3′-deoxythymidine, 2′,3′-dideoxyinosine, 2′,3′-dideoxy-3′-thiacytidine, 2′,3′-didehydro-2′,3′-dideoxythymidi-ne and the monophosphate nucleotides of 3′-azido-3′-deoxythymidine, 2′,3′-dideoxy-3′-thiacytidine and 2′,3′-didehydro-2′,3′-dide-oxythymidine.

Also contemplated herein are locked nucleic acid (LNA) nucleotides (e.g., 2′-O, 4′-C-methylene-(D-ribofuranosyl) nucleotides); 2′-methoxyethoxy (MOE) nucleotides; 2′-methyl-thio-ethyl, 2′-deoxy-2′-fluoro nucleotides, 2′-deoxy-2′-chloro nucleotides, 2′-azido nucleotides, and 2′-O-methyl nucleotides. Chemical modifications also include unlocked nucleic acids, or UNAs, which are non-nucleotide, acyclic analogues, in which the C2′-C3′ bond is not present.

Chemical modifications also include terminal modifications on the 5′ and/or 3′ part of the nucleic acids, and are also known as capping moieties. Such terminal modifications are selected from a nucleotide, a modified nucleotide, a lipid, a peptide, and a sugar. Chemical modifications also include “six membered ring nucleotide analogs.” Examples of six-membered ring nucleotide analogs include hexitol and altritol nucleotide monomers.

Chemical modifications also include “mirror” nucleotides which have a reversed chirality as compared to normal naturally occurring nucleotide. Mirror nucleotides may further include at least one sugar or base modification and/or a backbone modification. Mirror nucleotides include, for example, L-DNA (L-deoxyriboadenosine-3′-phosphate (mirror dA); L-deoxyribocytidine-3′-phosphate (mirror dC); L-deoxyriboguanosine-3′-phosphate (mirror dG); L-deoxyribothymidine-3′-phosphate (mirror image dT)) and L-RNA (L-riboudenosine-3′-phosphate (mirror rA); L-ribocytidine-3′-phosphate (mirror rC); L-riboguanosine-3′-phosphate (mirror rG); and L-ribouracil-3′-phosphate (mirror dU).

In some embodiments, modified ribonucleotides include modified deoxyribonucleotides, for example 5′OMe DNA (5-methyl-deoxyriboguanosine-3′-phosphate); PACE (deoxyriboadenosine 3′ phosphonoacetate, deoxyribocytidine 3′ phosphonoacetate, deoxyriboguanosine 3′ phosphonoacetate, deoxyribothymidine 3′ phosphonoacetate).

Nucleobases of the nucleic acids disclosed herein (such as small complementary nucleic acids, scRNAs and/or siRNAs) may include unmodified deoxyribonucleotides and ribonucleotides (purines and pyrimidines) such as adenine, guanine, cytosine, thymidine, and uracil. Nucleobases can be modified with natural and synthetic nucleobases, such as thymine, xanthine, hypoxanthine, inosine, 2-aminoadenine, 6-methyl and other alkyl derivatives of adenine and guanine, any “universal base” nucleotides; 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 8-halo, amino, thiol, thioalkyl, hydroxyl and other 8-substituted adenines and guanines, 5-trifluoromethyl and other 5-substituted uncials and cytosines, 7-methylguanine, deazapurines, heterocyclic substituted analogs of purines and pyrimidines, e.g., aminoethoxy phenoxazine, derivatives of purines and pyrimidines (e.g., 1-alkyl-, 1-alkenyl-, heteroaromatic- and 1-alkynyl derivatives) and tautomers thereof, 8-oxo-N6-methyladenine, 7-diazaxanthine, 5-methylcytosine, 5-methyluracil, 5-(1-propynyl)uracil, 5-(1-propynyl) cytosine and 4,4-ethanocytosine). Other examples of suitable bases include non-purinyl and non-pyrimidinyl bases such as 2-aminopyridine and triazines.

Sugar moieties in nucleic acids disclosed herein (such as small complementary nucleic acids, scRNAs and/or siRNAs) may include 2′-hydroxyl-pentofuranosyl sugar moiety without any modification. Alternatively, sugar moieties can be modified such as, 2′-deoxy-pentofuranosyl sugar moiety, D-ribose, hexose, modification at the 2′ position of the pentofuranosyl sugar moiety such as 2′-O-alkyl (including 2′-O-methyl and 2′-O-ethyl). i.e., 2′-alkoxy, 2′-amino, 2′-O-allyl, 2′-S-alkyl, 2′-halogen (including 2′-fluoro, chloro, and bromo), 2′-methoxyethoxy, 2′-O-methoxyethyl, 2′-O-2-methoxyethyl, 2′-propargyl, 2′-propyl, ethynyl, ethenyl, propenyl, CF, cyano, imidazole, carboxylate, thioate.

In some embodiments, the pentafuranosyl ring may be replaced with acyclic derivatives lacking the C2′-C3′-bond of the pentafuranosyl ring. For example, acyclonucleotides may substitute a 2-hydroxyethoxymethyl group for-the 2′-deoxyribofuranosyl sugar normally present in dNMPs.

The nucleoside subunits of the nucleic acids disclosed herein (such as small complementary nucleic acids, scRNAs and/or siRNAs) may be linked to each other by phosphodiester bond. The phosphodiester bond may be optionally substituted with other linkages. For example, phosphorothioate, thiophosphate-D-ribose entities, triester, thioate, 2′-5′ bridged backbone, PACE, 3′- (or -5′)deoxy-3′- (or -5′)thio-phosphorothioate, phosphorodithioate, phosphoroselenates, 3′- (or -5′)deoxy phosphinates, borano phosphates, 3′- (or -5′)deoxy-3′-(or 5′-)amino phosphoramidates, hydrogen phosphonates, phosphonates, borano phosphate esters, phosphoramidates, alkyl or aryl phosphonates and phosphotriester modifications such as alkylphosphotriesters, phosphotriester phosphorus linkages, 5′-ethoxyphosphodiester, P-alkyloxyphosphotnester, methylphosphonate, and nonphosphorus containing linkages for example, carbonate, carbamate, silyl, sulfur, sulfonate, sulfonamide, formacetal, thioformacetyl, oxime, methyleneimino, methylenemethylimino, methylenehydrazo, methylenedimethylhydrazo and methyleneoxymethylimino linkages.

Nucleic acid molecules disclosed herein (such as small complementary nucleic acids, scRNAs and/or siRNAs) may include a peptide nucleic acid (PNA) backbone. The PNA backbone is includes repeating N-(2-aminoethyl)-glycine units linked by peptide bonds. The various bases such as purine, pyrimidine, natural and synthetic bases are linked to the backbone by methylene carbonyl bonds.

Modifications can be made at terminal phosphate groups. Non-limiting examples of different stabilization chemistries can be used, e.g., to stabilize the 3′-end of nucleic acid sequences, including (1) [3-3′]-inverted deoxyribose; (2) deoxyribonucleotide; (3) [5′-3′]-3′-deoxyribonucleotide; (4) [5′-3′]-ribonucleotide; (5) [5′-3′]-3′-O-methyl ribonucleotide; (6) 3′-glyceryl; (7) [3′-5′]-3′-deoxyribonucleotide; (8) [3′-3′]-deoxyribonucleotide; (9) [5′-2′]-deoxyribonucleotide; and (10) [5-3′]-dideoxyribonucleotides. In addition to unmodified backbone chemistries can be combined with one or more different backbone modifications described herein.

Modified nucleotides and nucleic acid molecules as provided herein may include conjugates, for example, a conjugate covalently attached to a nucleic acid molecule. The conjugate may be covalently attached to a nucleic acid molecule via a linker. In one embodiment, a conjugate molecule may include a molecule that facilitates delivery of a nucleic acid molecule into a cell, such as, for example, into a particular type of cell, or into a particular intracellular compartment or vesicle within a cell. The conjugate may also comprise, for example, a detectable label, or a molecule that facilitates binding or detection by a second molecule.

In some embodiments, the small complementary nucleic acids, such as scRNAs, may be about 500 or less, about 400 or less, about 300 or less, about 200 or less, about 100 or less, about 90 or less, about 80 or less, about 70 or less, about 60 or less, about 50 or less, about 40 or less, about 30 or less, about 25 or less, about 20 or less, about 15 or less, about 14 or less, about 13 or less, about 12 or less, about 11 or less, about 10 or less, about 9 or less, about 8 or less, about 7 or less, about 6 or less, about 5 or less, about 4 or less, about 3 or less, or about 2 or less, bases in length.

In some embodiments, the small complementary nucleic acid (such as scRNA) of the present disclosure comprises, consists of, or contains a sequence that has a particular sequence identity to a sequence complementary to an intron sequence of interest. In some embodiments, the sequence has at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99%, sequence identity to a sequence complementary to an intron sequence of interest.

In some embodiments, the small complementary nucleic acid (such as scRNA) of the present disclosure comprises at sequence having at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99%, sequence identity to the sequence 5′-cacgccugugaaaccguacuaagucucccgugucuucuuaucaccaucag-3′.

Combining Small Complementary Nucleic Acids and siRNA

In conjunction, in admixture, in combination, in series, or simultaneously, siRNAs can be used with small complementary nucleic acids (such as scRNA) of the present disclosure. Small complementary nucleic acids (such as scRNA) of the present disclosure target an intron, preferably by full- or partial complementary base-pairing (hybridization) thereto. In some embodiments, the small complementary nucleic acids (such as scRNA) of the present disclosure hybridize to a Py-containing part of intron 4 of the MIE gene of HCMV in the nucleus. In contrast, siRNA may be used that targets the mRNA (e.g., of the MIE gene of HCMV) in the cytoplasm, to form RISC complexes and cause degradation of the mRNA. Since the underlying mechanisms of siRNA and the small complementary nucleic acids (such as scRNA) of the present disclosure are different, additive or synergistic inhibitory effects on viral gene expression and replication may be obtained in embodiments herein.

The siRNA and the small complementary nucleic acids (such as scRNA) of the present disclosure may be delivered at the same time, or at different times. The siRNA and the small complementary nucleic acids (such as scRNA) of the present disclosure may be delivered using the same delivery mechanism or vehicle, or using different delivery mechanisms or vehicles.

### Delivery of Therapeutic Products and Compositions

Delivery of nucleic acids of the present disclosure, such as the small complementary nucleic acids (such as scRNA) and/or siRNA, may be achieved by a number of methods known to one of ordinary skill in the art. In some embodiments, they can be directly introduced into cells. Non-limiting methods to introduce these components into a cell include microinjection, electroporation, nucleofection, lipofection, particle gun technology, and microprojectile bombardment.

Delivery of nucleic acids of the present disclosure, such as the small complementary nucleic acids (such as scRNA) and/or siRNA, may also be achieved by using a delivery vehicle, such as a particle, lipid vesicle or carrier. Nucleic acid molecules may be delivered or administered to a subject by direct application of the agent (such as a nucleic acid molecule) with a carrier or diluent or any other delivery vehicle that acts to assist, promote or facilitate entry into a cell, including viral sequences, viral particular, liposome formulations, lipofectin or precipitating agents and the like.

Delivery of nucleic acids of the present disclosure, such as the small complementary nucleic acids (such as scRNA) and/or siRNA, may also be achieved by a variety of methods known to those of skill in the art, including, but not limited to, encapsulation in liposomes, by iontophoresis, or by incorporation into other vehicles, such as biodegradable polymers, hydrogels, cyclodextrins, poly(lactic-co-glycolic)acid (PLGA) and PLCA microspheres, biodegradable nanocapsules, and bioadhesive microspheres, or by proteinaceous vectors.

Nucleic acids of the present disclosure, such as the small complementary nucleic acids (such as scRNA) and/or siRNA, can be complexed with cationic lipids, packaged within liposomes, or otherwise delivered to target cells or tissues. The nucleic acid or nucleic acid complexes can be locally administered to relevant tissues ex vivo, or in vivo through direct dermal application, transdermal application, or injection, with or without their incorporation in biopolymers. Delivery systems include surface-modified liposomes containing poly(ethylene glycol) lipids (PEG-modified, or long-circulating liposomes or stealth liposomes). These formulations offer a method for increasing the accumulation of drugs in target tissues.

Nucleic acids of the present disclosure, such as the small complementary nucleic acids (such as scRNA) and/or siRNA, can be formulated or complexed with polyethylenimine (e.g., linear or branched PEI) and/or polyethylenimine derivatives, including for example polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives, grafted PEIs such as galactose PEI, cholesterol PEI, antibody derivatized PEI, and polyethylene glycol PEI (PEG-PEI), for example.

In some embodiments, nucleic acids of the present disclosure, such as the small complementary nucleic acids (such as scRNA) and/or siRNA, are formulated within delivery particles, including, but not limited to, nanoparticles. In some embodiments, small complementary nucleic acids (such as scRNA) and siRNA are formulated within the same nanoparticles. In other embodiments, small complementary nucleic acids (such as scRNA) and siRNA are formulated within different, and/or separate, nanoparticle(s).

In some embodiments, particles (such as nanoparticles) comprise one or more cationic polymers. In some embodiments, the one or more polymers may comprise a polyamine or polyimine, or derivative thereof.

In some embodiments, the particles may comprise a core and one or more shell layers encompassing the core. In some embodiments, a particle, or particle core, may comprise one or more nucleic acids complexed with polyethylene imine (PEI) and polyspermine-imidazole-4,5-imine (PSI). In some embodiments, networked cationic polymer, which may be used as a particle or particle core (e.g., in the range of 100-400 nm) are prepared by condensation of spermine imidazole-4,5-aldehyde oligomer and branched polyethyleneimine (PEI-800).

In some embodiments, the size of a networked cationic polymer can be adjusted by varying the pH of the solution (e.g., in which such a condensation reaction occurs), and/or by adjusting the salt concentration. In some embodiments, a pH of about 9-12 is used, and/or a salt concentration of about 0-30 mM is used. In some embodiments, a pH of about 9 is used. In some embodiments, the salt is sodium chloride.

In some embodiments, a core particle is wrapped with at least one wrapping layer. In some embodiments, the wrapping layer is a polymer. In some embodiments, the polymer is a hydrophilic and/or water-soluble polymer. In some embodiments, the wrapping polymer is a polyethylene glycol (PEG).

In some embodiments, particles may be specifically targeted to particular destinations, including, for example, particular cells or cell types, using a targeting moiety. In some embodiments, the targeting moiety is a molecule, ligand, or protein, that binds to a molecule on a surface of a target cell. In some embodiments, particles may comprise a targeting moiety that preferentially targets the particles to HCMV-infected cells (or cells infectable by HCMV). In some embodiments, the targeting moiety is a chemokine molecule. In some embodiments, the chemokine molecule is CX3CL1, or a mutant or derivative thereof that binds the US28 receptor expressed on the surface of HCMV-infected cells.

A targeting moiety may be conjugated to a particle using various techniques known to persons of skill in the art.

In some embodiments, a targeting moiety may be conjugated to lysine to generate an extra amino group (e.g., at or toward the N- and/or C-terminus). In some embodiments, lysine is conjugated to the C-terminus of a targeting moiety, such as CX3CL1, or a mutant or derivative thereof. In some embodiments, the targeting moiety is immobilized to a particle, core, or wrapping layer, by conjugation. In some embodiments, the targeting moiety (such as CX3CL1) is immobilized to a PEG wrapping layer, such as by using a free or introduced amino group in the targeting moiety.

In some embodiments, the nucleic acid-containing particles, such as particles containing a nucleic acid-containing core and one or more wrapping layers, as described herein, are taken up by cells (such as HCMV-infected cells) into endosomes, and the nucleic acid is releasable from the core complex (to gain entry into the cytosol) under the low-pH conditions of the endosome.

In some embodiments, nucleic acids of the present disclosure, such as the small complementary nucleic acids (such as scRNA) and/or siRNA, can be complexed with membrane disruptive agents. The membrane disruptive agent or agents and the nucleic acid molecule may also be complexed with a cationic lipid or helper lipid molecule.

Delivery systems may include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone).

Compositions, methods and kits disclosed herein may include an expression vector that includes a nucleic acid sequence encoding at least one nucleic acid molecule of the invention in a manner that allows expression of the nucleic acid molecule. Methods of introducing nucleic acid molecules or one or more vectors capable of expressing the nucleic acid molecule in the environment of the cell will depend on the type of cell and the make up of its environment.

In some embodiments, nucleic acids of the present disclosure, such as the small complementary nucleic acids (such as scRNA) and/or siRNA, may be expressed from transcription units inserted into DNA or RNA vectors. Recombinant vectors can be DNA plasmids or viral vectors. Nucleic acid molecule-expressing viral vectors can be constructed based on, but not limited to, adeno-associated virus, retrovirus, adenovirus, or alphavirus. The recombinant vectors capable of expressing the nucleic acid molecules can be delivered as described herein, and persist in target cells. Alternatively, viral vectors can be used that provide for transient expression of nucleic acid molecules. Such vectors can be repeatedly administered as necessary. Delivery of nucleic acid molecule-expressing vectors can be systemic, such as by intravenous or intramuscular administration.

An expression vector may include one or more of the following: a) a transcription initiation region (e.g., eukaryotic pol I, II or III initiation region); b) a transcription termination region (e.g., eukaryotic pol I, II or III termination region); c) an intron and d) a nucleic acid sequence encoding at least one of the nucleic acid molecules, wherein said sequence is operably linked to the initiation region and the termination region in a manner that allows expression and/or delivery of the nucleic acid molecule.

Transcription of the nucleic acid molecule sequences can be driven from a promoter for eukaryotic RNA polymerase I (pol I). RNA polymerase II (pol II), or RNA polymerase III (pol III).

The useful dosage of nucleic acids herein to be administered, and the particular mode of administration, will vary depending upon such factors as the cell type, or for in vivo use, the age, weight and the particular animal and region thereof to be treated, the particular agent (e.g., a nucleic acid) and delivery method used, the therapeutic or diagnostic use contemplated, and the form of the formulation, for example, suspension, emulsion, micelle or liposome, as will be readily apparent to those skilled in the art.

When lipids are used to deliver the nucleic acid, the amount of lipid compound that is administered can vary and generally depends upon the amount of nucleic acid being administered. For example, the weight ratio of lipid compound to nucleic acid is preferably from about 1:1 to about 30:1, with a weight ratio of about 5:1 to about 10:1 being more preferred.

A suitable dosage unit of nucleic acid molecules may be in the range of 0.001 to 0.25 milligrams per kilogram body weight of the recipient per day, or in the range of 0.01 to 20 micrograms per kilogram body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day.

The nucleic acids of the present disclosure, such as the small complementary nucleic acids (such as scRNA) and/or siRNA, can be used to treat individuals infected with, or suspected of being infected with, a herpesvirus or adenovirus, such as a herpesvirus like HCMV.

The administration of the nucleic acids of the present disclosure, such as the small complementary nucleic acids (such as scRNA) and/or siRNA, may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. The agents or compositions can be administrated in one or more doses. In some embodiments, an effective amount is administrated as a single dose. In other embodiments, an effective amount is administrated as more than one dose, over a period time. The determination of optimal ranges of effective amounts of a given cell type for a particular disease or condition is within the skill of those in the art.

Treatments of the present disclosure can be provided in conjunction with, before, or after, other anti-herpesvirus or anti-adenovirus treatments, including, for example, nucleoside analogies, synthetic acyclic analogues of 2′-deoxy-guanosine (and their derivatives), and low molecular weight compounds that target the CMV terminase complex.

### Experimental

Non-limiting embodiments of the present invention are illustrated in the following Examples. Efforts have been made to ensure accuracy with respect to numbers used (e.g., amounts, concentrations, percent changes, and the like), but some experimental errors and deviations should be accounted for.

Unless indicated otherwise, temperature is in degrees Centigrade and pressure is at or near atmospheric. It should be understood that these Examples are given by way of illustration only and are not intended to limit the scope of what the inventor regards as various embodiments of the present invention. Not all of the following steps set forth in each Example are required nor must the order of the steps in each Example be as presented.

### Example 1

Identification of the Py that is Responsible for Transcriptional Switching from IE1 to IE2

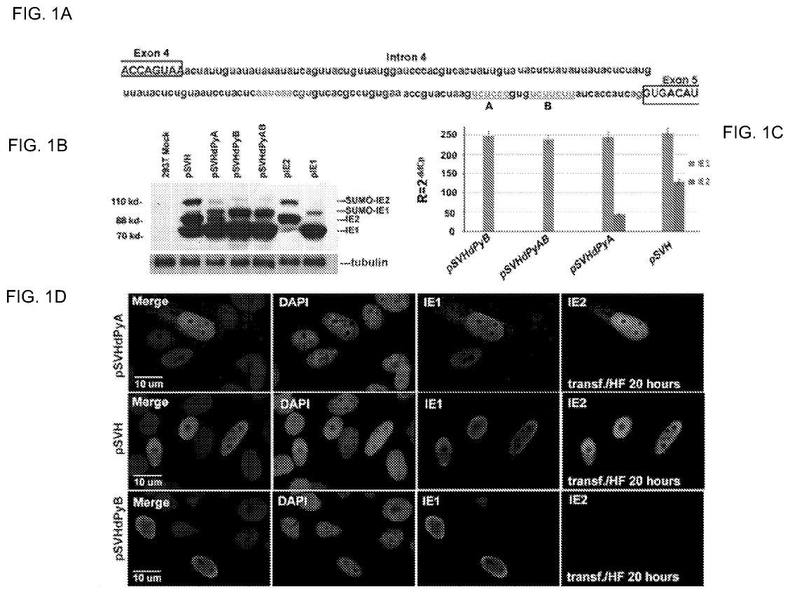

At the end of exon 4 (FIG. 1A), there exists stop codons and polyA signals for IE1 gene expression. Additionally, as also shown in FIG. 1A, there are 2 Py sequences (ucuccc (A) and ucuucuu (B)) between exons 4 and 5, and after the polyA signal of IE1. To elucidate how the switch from IE1 to IE2 is regulated, and in particular, whether the DNA sequence after the IE1 polyA signals could be important for gene splicing of IE2, mutations in Py sequences were created.

Using the pSVH plasmid, three different mutations were created: pSVHdPyA (in which the ucuccc was mutated to ucagcc); pSVHdPyB (in which the ucuucuu was mutated to ucaaguu); and pSVHdPyAB (in which both A and B were mutated). After transfecting the plasmids into HEK 293T cells, Western blot assay (FIG. 1B) and real-time RT PCR assay (FIG. 1C) were used to examine the production of IE1 and of IE2 at both the protein level and transcriptional level, respectively. It was determined thereby that both pSVHdPyB and pSVHdPyAB failed to express IE2 at any appreciable level, although the expression of IE1 was not affected. It was also observed that pSVHdPyA expressed IE2 at a much lower level than pSVH did. The results indicated that: the second Py (B) was required for the gene splicing to produce IE2 (because its mutation (ucaaguu) failed to express IE2); and that the first Py (A) is important for IE2 splicing and expression.

Further, immunofluorescent assay (IFA) was performed to examine the production of IE1/2 from transfected MRC-5 cells. MRC-5 cells were transfected with pSVHdPyA (upper panel of FIG. 1D), pSVH (middle panel of FIG. 1D), or pSVHdPyB (lower panel of FIG. 1D), for 20 hours. The cells were then fixed with 1% paraformaldehyde, and permeabilized with 0.2% triton X-100. The cells were then incubated with anti-IE1 antibody, or anti-IE2 antibody, to show the production of IE1 or IE2, respectively. Total cells were visualized using DAPI staining. All colors were merged in the first column.

As shown in FIG. 1D, IE1 production was not discernably affected, as it was detected in the cells irrespective of the plasmid used for transfection. However, no IE2 production was detected in the cells transfected with pSVHdPyB.

### Example 2

### The Polypyrimidine Tract (Py) Mediates the Interaction Between Intron and Splicing Factors

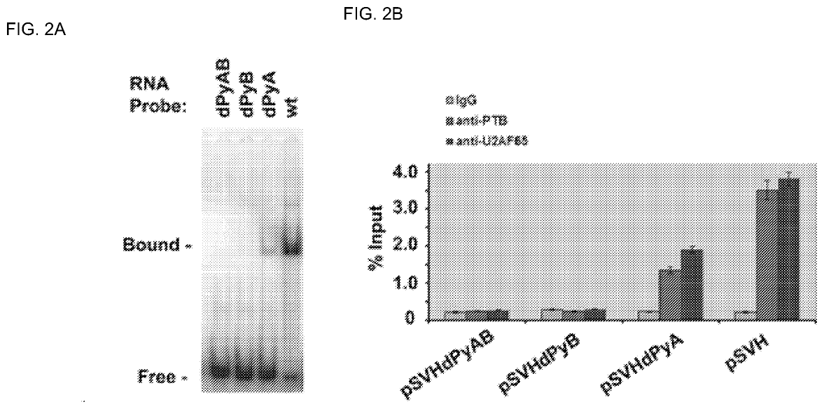

To determine whether PyA or PyB mediates the interaction between intron 4 and U2AF65, RNA EMSA assays were performed. Specifically, U2AF65 protein was incubated with radiolabeled RNA probes (FIG. 2A; sequences shown in Table 1), and resolved using native PAGE in the following lanes: 1) RNA without the Pys A and B (dPyAB); 2) RNA without PyB (dPyB); 3) RNA without PyA (dPyA); and 4) RNA with both Pys A and B (wt). As can be seen by comparing the bound probes to that in wt (lane 4), it was determined that U2AF65 failed to bind to the RNA probe in which PyB was mutated (first and second lanes); and that the binding became much weaker when PyA was mutated (third lane).

TABLE 1
scRNA sequences targeting the HCMV MIE gene

| Intron | |
|---|---|
| Number | Nucleotide Sequence |
| Intron 1 | 5′ GUGUCAAGGACGGUGACUGCAGAAAGACCCAUGGAAAGG |
| Intron 2 | 5′ CCUAAACACAUGUGAAAUAGUGUCAUAAGCACAUGGGUCACA |
| Intron 3 | 5′ GUAGAGGAGGAUAACAACACAUAUAAGUAUCCGUCCACC |
| Intron 4 | 5′ CUGAUGGUGAUAAGAAGACACGGGAGACUUAGUACGGUUUCACAGGCGU |

Further, to determine whether the Pys could affect U2AF-intron 4 binding in vivo, an RNA ChIP assay was performed to determine whether splicing regulators (U2AF65 and PTB) could interact with Py. Specifically, HEK 293T cells were transfected with 1 of the 4 different plasmids (FIG. 2B) for 24 hours, cross-linked with 1% paraformaldehyde, and then sheared with a sonicator to break up the RNA. RNA-protein complexes were pulled-down using control IgG, anti-PTB, and anti-U2AF65 antibodies. After being washed multiple times, the cross-linking was reversed with SDS at 95° C. The DNA was removed by DNase I digestion, and the RNA was purified through the column provided in the RNA ChIP kit. Real-time RT-PCR was performed to determine the amount of specific RNA in each ChIPed sample, and the percentage of RNA was calculated by comparing it to the input RNA. The primers used for the RT-PCR are shown in Table 2.

TABLE 2
Primers for PCR to detect the HCMV MIE gene

| Primer Name | Primer Sequence |
|---|---|
| pShort | 5′ ATG TCC TGG CAG AAC 3' |
| plE1 | 5′ CAT CCT CCC ATC ATA TTA 3' |
| plE2 | 5′-GGA TGC CCC GGG GAG AGG-3' |

As can be seen in FIG. 213, when PyB was mutated, the intron RNA lost the ability to bind to U2AF65 or PTB. When PyA was mutated, the intron RNA's binding to PTB or U2AF was significantly weaker than that of wild type.

Accordingly, it was determined from these experiments that both Pys in intron 4 are important if either splicing factor is to bind to intron 4 to facilitate IE2 gene splicing. The second Py (PyB) is essential for the interaction of the splicing factors with intron, while the first Py (PyA) is important for their interaction.

### Example 3

### PyA and PyB Affect IE2 Gene Splicing

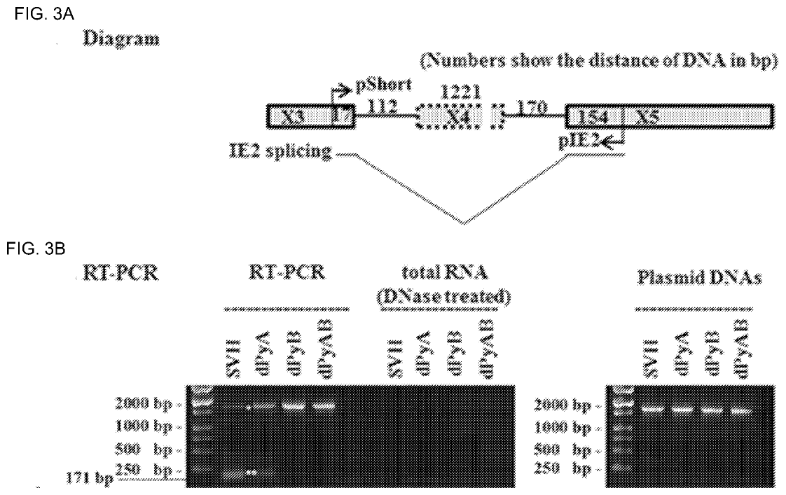

To determine whether PyA or PyB is important for IE2 gene splicing, an in vivo gene-splicing assay was performed. First, plasmids (pSVH, pSVHdPyA, pSVHdPyB, or pSVHdPyAB) were transfected into HEK 293T cells for 20 hours, and total RNA was thereafter harvested. The total RNA was treated with DNase to remove any contaminating plasmid DNA. Subsequently, RT-PCR was performed using a specific reverse primer (pIE2) in exon 5 (Table 2), instead of oligodT for reverse transcription (RT), so that both mRNA and pre-mRNA could be reverse transcribed (FIG. 3A). By doing so, it was possible to amplify cDNA on both pre-mRNA (*) and mRNA (**) using a forward primer in exon 3 and a reverse primer (pIE2) in exon 5 (Table 2). PCR was performed using the total RNA (DNase treated) as templates.

As shown in the middle panel of FIG. 3B, no DNA could be amplified, excluding the possibility of contamination by plasmid DNA in the total RNA preparation. Additionally, a PCR experiment using plasmid DNA as template amplified a DNA of a larger size (right side of FIG. 3B).

As can be seen from the left side of FIG. 3B, RT-PCR resulted in 2 bands for the pSVH- and pSVHdPyA-transfected groups, which represent the amplified DNA from pre-mRNA (*) and mRNA (**), respectively. The mRNA band was not detectable for the pSVHdPyB and pSVHdPyAB groups. It can also be seen that the mRNA band from the pSVHdPyA-transfected group was much weaker than that from the pSVH-transfected group. These results demonstrate that PyB is required, and PyA is important, for IE2 splicing as well as efficient production of mRNA from abundant pre-mRNA.

### Example 4

### PyB is Essential and PyA is Important for HCMV Replication

Mutations in the Py were introduced into HCMV BACmid, resulting in the following BACmids: HB5dPyA, HB5dPyB, and HB5dPyAB. To determine whether the BACmid could express IE1/2 after transfection into MRC-5 cells, MRC-5 cells were fixed at 20 hours post-transfection, and IFA was performed to examine the IE1/2 production.

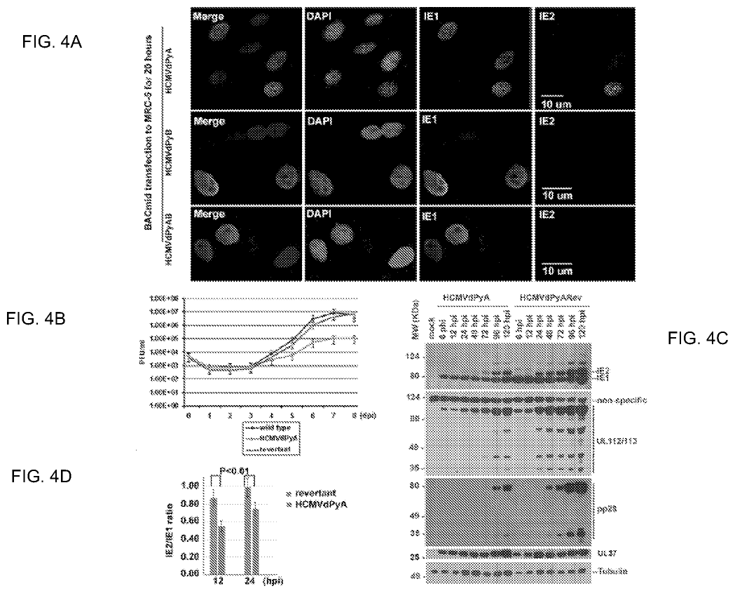

As shown in FIG. 4A, only the BACmid HCMVdPyA produced both IE1 and IE2, while the other two BACmids only produced IE1, and not IE2. This result was consistent with that shown in FIG. 1 (after transfection with the plasmids). Additionally, revertant BACs were generated (HB5dPyARev, HB5dPyBRev, and HB5dPyABRev).

Each BACmid was transfected into MRC-5 cells to allow virus formation. HB5dPyA was able to form infectious virus particles. However, the other 2 BACmids (HB5dPyB and HB5dPyAB) failed to yield any infectious virus. All the revertant BACmids were able to make infectious virus, and they also replicated similarly to wild type.

By comparing the viral replication of HCMVdPyA with that of wild-type HCMV (or its revertant), it was determined that HCMVdPyA had a defective phenotype in replication (FIG. 4B). Western blot assay also showed that viral protein production was reduced when the first Py is mutated (FIG. 4C). As expected, IE2 protein was produced much later in the HCMVdPyA than that in the revertant HCMV. Accordingly, it was determined that the first Py sequence is important for HCMV replication, while the second Py is required for HCMV replication.

FIGS. 1 and 4A demonstrate that in a transfection system, PyA is important for IE2 production. To confirm these results in a viral infection system, human fibroblast cells (MRC-5) were infected with HCMVdPyA (or its revertant) for different times, and the cells were fixed at 12 hpi and 24 hpi for immunostaining (using antibodies against IE1 (FITC) and IE2 (Texas red)). IE1- and IE2-positive cells were counted under fluorescence microscopy.

At 12 hpi, only 55 IE2-positive cells were seen in every 100 IE1-positive cells when infected with HCMVdPyA, compared to the 87 IE2-positive cells that were seen in every 100 IE1-positive cells in the revertant virus infection (FIG. 4D). At 24 hpi, the IE2/IE1 ratio was closer to 1 (74% for HCMVdPyA infection to 99% for its revertant infection). These results demonstrated that PyA was important for IE2 production in a viral infection system.

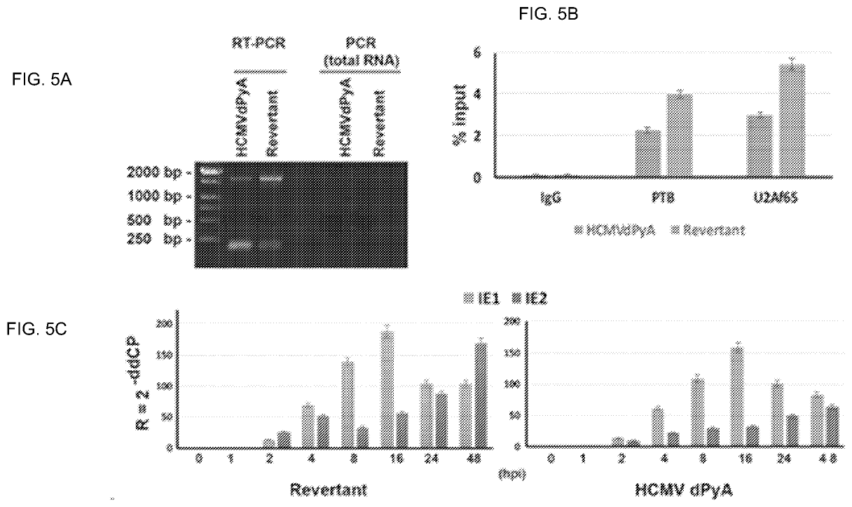

Additionally, to determine whether IE2 gene splicing is affected when PyA is mutated, in the context of viral infection, MRC-5 cells were infected with HCMVdPyA (or its revertant) at an MOI of 0.1 for 16 hours. Total RNA was isolated, and treated with DNase I. As shown at the right side of FIG. 5A, the PCR was negative when using the total RNA as the template, excluding the possibility of viral DNA contamination. The left side of FIG. 5A shows the RT-PCR products amplified from the mRNA (171 bp bands) or from pre-mRNA (1.7 kb). It can be seen that the IE2 splicing is stronger in the revertant infection than that in HCMVdPyA infection, since the mRNA band from the revertant infection is significantly more intense than that from HCMVdPyA infection. Therefore, it was determined that the mutation of PyA negatively affected IE2 gene splicing.

Additionally, RNA ChIP assay was performed to determine whether PyA played a role in the interaction of splicing factors with intron 4 of MIE. MRC-5 cells were fixed after infection with HCMVdPyA (or its revertant) for 16 hours at an MOI of 1. RNA ChIP assays were performed as described above using anti-PTB or anti-U2AF65 antibodies. As shown in FIG. 5B, both PTB and U2AF65 interacted with intron 4 of IE2 RNA in the cells that were infected with HCMVdPyA or its revertant. However, the interaction was reduced when the PyA was mutated. The results thereby confirmed that PyA enhanced the interaction of splicing factors with intron 4.

The results shown in FIG. 4C demonstrate that mutation of PyA reduces viral gene expression at the translational level. To determine if PyA affects IE1/IE2 gene expression at the mRNA level, real-time RT-PCR was performed to determine the IE1/IE2 mRNA level at different times after infection of MRC-5 cells with HCMVdPyA (or its revertant) at an MOI of 0.5.

As can be seen in FIG. 5C, IE1 mRNA level remained at a comparable level from HCMVdPyA infected cells, but the IE2 mRNA level was significantly decreased in the PyA-mutated viral infections. Accordingly, these results demonstrated that PyA was not required for HCMV to replicate, but is important to enhance the interaction of splicing factors with intron 4 (and strengthen viral gene expression and viral replication).

### Example 5

### Design and Synthesis of an Intron-Targeting Small Complementary Nucleic Acid

The present inventors thus conceived of an antiviral mechanism in which small nucleic acids (such as RNA), complementary to an intron containing one or more Pys, could be used as an antiviral agent to virus replication (such as HCMV replication), by abolishing the interaction of splicing factor (such as U2AF65) with Py, thereby leading to inhibition of gene splicing.

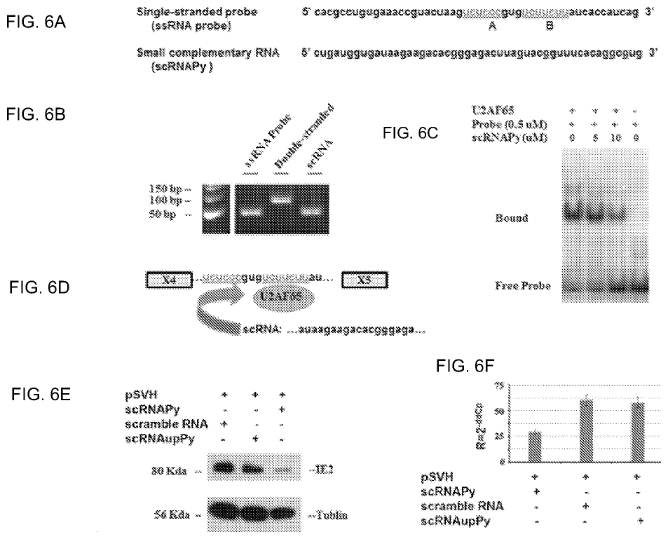

To demonstrate this effect with HCMV, an IE2 gene model was used. First, a small RNA (scRNAPy) complementary to an RNA probe that was shown to interact with U2AF65 was produced. FIGS. 2A and 6A. It was demonstrated that this scRNAPy and the probe RNA formed double-stranded RNA when they were mixed in a 1:1 molar ratio (for 30 min at room temperature). The single-stranded RNA band and the double-stranded RNA band are shown in FIG. 6B.

Additionally, an RNA gel shift assay was performed in the absence or the presence of the scRNAPy. As shown in FIG. 6C, when the scRNAPy was added to the probe-U2AF65 reaction system, the binding of the probe with U2AF65 was reduced, and the reduction of the probe-U2AF65 binding was associated with the amount of scRNAPy.

To determine whether the interference of scRNAPy with the binding of U2AF65 and the Py tract could occur in vivo (to repress IE2 gene splicing), as depicted in FIG. 6D, HEK 293T cells were co-transfected with the pSVH together with the scRNAPy, with the scRNAPyup (an scRNA that is complementary to the upstream of intron 4), or with a scrambled RNA (made from luciferase cDNA), for 20 hours. Whole cell lysate samples were then prepared for use in a Western blot assay to check for IE2 protein production. Additionally, total RNA samples were prepared for use in real-time PCR, to examine the mRNA levels of IE2. As can be seen in FIGS. 6E and 6F, the scRNAPy significantly reduced IE2 expression at both the protein level and the transcriptional level. The results indicate that an scRNAPy, for example, can effectively interfere with IE2 gene splicing and expression.

1. A single-stranded nucleic acid molecule comprising a sequence complementary to all or a part of an intron sequence of a viral mRNA containing one or more polypyrimidine (Py) tracts, wherein said nucleic acid molecule is capable of hybridizing to a region of said intron sequence containing one or more of said polypyrimidine (Py) tracts.

2. The nucleic acid molecule of claim 1, wherein said viral mRNA is from the HCMV MIE gene.

3. The nucleic acid molecule of claim 2, wherein the intron sequence is the fourth intron of the HCMV MIE gene.

4. The nucleic acid molecule of claim 3, wherein said nucleic acid molecule, when introduced into HCMV-infected cells, is capable of inhibiting splicing and expression of HCMV IE2.

5. The nucleic acid molecule of claim 1, wherein said sequence complementary to all or a part of the intron sequence is between 10-100 nucleotide in length.

6. The nucleic acid molecule of claim 5, wherein said sequence complementary to all or a part of the intron sequence is between 10-50 nucleotides in length.

7. The nucleic acid molecule of claim 1, wherein said sequence complementary to all or a part of the intron sequence comprises the sequence 5′-cacgccugugaaaccguacuaagucucccgugucuucuuaucaccaucag-3′.

8. The nucleic acid molecule of claim 1, wherein said sequence complementary to all or a part of the intron sequence comprises a sequence having 90% or more sequence identity to the sequence 5′-cacgccugugaaaccguacuaagucucccgugucuucuuaucaccaucag-3′.

9. A composition comprising the nucleic acid molecule of claim 1, wherein said composition further comprises a delivery vehicle.

10. The composition of claim 9, wherein the delivery vehicle comprises a polymer.

11. The composition of claim 10, wherein the polymer comprises a cationic polymer.

12. The composition of claim 10, wherein the delivery vehicle is a delivery particle.

13. The composition of claim 12, wherein said delivery particle comprises a core structure comprising said nucleic acid molecule and at least one polymer, and wherein said delivery particle further comprises at least one wrapping layer that envelops said core structure.

14. The composition of claim 13, wherein said core structure comprises said nucleic acid molecule, polyethylene imine (PEI), and polyspermine-imidazole-4,5-imine (PSI).

15. The composition of claim 13, wherein said at least one wrapping layer comprises a hydrophilic polymer.

16. The composition of claim 15, wherein said hydrophilic polymer is polyethylene glycol.

17. The composition of claim 12, wherein said delivery particle comprises a targeting moiety.

18. The composition of claim 17, wherein said targeting moiety targets said delivery particle to HCMV-infected cells.

19. The composition of claim 18, wherein said targeting moiety is CX3CL1, or a mutant or derivative thereof.

20. The nucleic acid of claim 1, wherein the nucleic acid molecule contains at least one of a modified base, a base analog, and an abasic site.

21. The nucleic acid of claim 1, wherein the nucleic acid molecule contains DNA, RNA, or DNA and RNA.

22. The nucleic acid of claim 21, wherein the nucleic acid molecule is a short complementary RNA (scRNA).

23. The nucleic acid molecule of claim 1, wherein the nucleic acid molecule is conjugated to a heterologous molecule.

24. A method for treating a herpesvirus infection, preferably HCMV infection, comprising administering to a subject in need thereof the nucleic acid molecule of claim 1.

25. A method for treating a herpesvirus infection, preferably HCMV infection, comprising administering to a subject in need thereof the composition of claim 9.

26. A method of inhibiting transcriptional switching, comprising contacting the nucleic acid of claim 1 with a viral mRNA.
